(19) United States
(12) Patent Application Publication
(10) Pub. No.: US 2016/0015638 A1
(43) Pub. Date: Jan. 21, 2016

(54) STABLE GLUCOKINASE ACTIVATOR COMPOSITIONS

(71) Applicant: vTv Therapeutics LLC, High Point, NC (US)

(72) Inventors: Yun Mo, Dix Hills, NY (US); Mahendra G. Dedhiya, Pomona, NY (US); Anil Chhettry, Holtsville, NY (US)

(21) Appl. No.: 14/840,682

(22) Filed: Aug. 31, 2015

Related U.S. Application Data
(63) Continuation of application No. PCT/US2014/019349, filed on Feb. 28, 2014.
(60) Provisional application No. 61/772,022, filed on Mar. 4, 2013.

(51) Int. Cl. A61K 9/14; A61K 31/426

(52) U.S. Cl. CPC A61K 9/145; A61K 31/426; A61K 9/146

(57) ABSTRACT

The invention relates to stable pharmaceutical compositions comprising a glucokinase (GK) activator suitable for oral administration. The invention also relates to methods of making and using such pharmaceutical compositions.

### FIELD OF THE INVENTION

The invention relates to stable pharmaceutical compositions comprising a glucokinase (GK) activator suitable for oral administration. The invention also relates to methods of making and using such pharmaceutical compositions.

### BACKGROUND OF THE INVENTION

{2-[3-cyclohexyl-3-(trans-4-propoxy-cyclohexyl)-ureido]-thiazol-5-ylsulfanyl}-acetic acid (disclosed in, for example, U.S. Pat. No. 7,851,636) is a GK activator that sensitizes the glucokinase (GK) sensor system. GK is an enzyme that belongs to the family of hexokinases, which catalyze the first step in the metabolism of glucose, i.e., conversion of glucose to glucose-6-phosphate. GK may play a role in regulating carbohydrate metabolism by acting as a glucose sensor and causing shifts in metabolism or cell function in response to fluctuating blood-glucose levels. GK functions as a glucose sensor in the pancreas, liver, gut and brain. {2-[3-cyclohexyl-3-(trans-4-propoxy-cyclohexyl)-ureido]-thiazol-5-ylsulfanyl}-acetic acid is a liver-selective GK activator that does not increase insulin secretion by the pancreas in the presence of glucose.

Ideal drugs for oral administration have moderate to high water solubility and membrane permeability, which quickly dissolve the drug in gastrointestinal fluids, as well as allow for quick absorption into the bloodstream. However, a significant amount of drug candidates are poorly soluble, and present a major hurdle for oral delivery. Poor solubility is often the reason for incomplete or erratic absorption, poor bioavailability, slow-onset of action, patient-to-patient PK variability, strong food effects and high dose requirements.

Formulation strategies have been explored to improve solubility of low solubility drugs, including forming solid dispersions of amorphous drugs, liquid filled capsules, and particle size reduction. Particle size reduction involves reducing larger drug particles to form smaller nanoparticles. However, forming drug nanoparticles has its challenges. For example, stabilizing nanoparticles from aggregation is difficult, particularly when formulating them into solid dosage forms. Conditions created during conversion of particle suspensions into solid forms can lead to particle aggregation, increases in particle size or induce crystallization of stabilizers, which present a great challenge in maintaining nanoparticle size and stability. Further, formation of particle aggregates is typically irreversible where agglomerates cannot revert back to individually dispersed particles once they are reconstituted in dispersing medium.

Certain GK activators are poorly soluble, including {2-[3-cyclohexyl-3-(trans-4-propoxy-cyclohexyl)-ureido]-thiazol-5-ylsulfanyl}-acetic acid, leading to high dose requirements, high PK variability and strong food effects. Thus there is a need for stable formulations that improve solubility and stability of pharmaceutical compositions containing such agents. Applicants have now developed such soluble, stable and bioavailable formulations, which are disclosed herein.

### SUMMARY OF THE INVENTION

The invention relates to stable pharmaceutical compositions comprising a glucokinase (GK) activator suitable for oral administration. The invention also relates to methods of making and using such pharmaceutical compositions.

In one aspect, the present invention relates to a pharmaceutical composition comprising nanoparticles and one or more alkalizers, wherein the nanoparticles have a mean diameter between about 0.5 nm to about 1000 nm, have a polydispersity index of about 0.001 to about 0.400 and comprise {2-[3-cyclohexyl-3-(trans-4-propoxy-cyclohexyl)-ureido]-thiazol-5-ylsulfanyl}-acetic acid or a pharmaceutically acceptable salt thereof.

In some embodiments, the present invention relates to a pharmaceutical composition comprising nanoparticles, one or more alkalizers and one or more redispersing agents, wherein the nanoparticles have a mean diameter between about 0.5 nm to about 1000 nm and comprise {2-[3-cyclohexyl-3-(trans-4-propoxy-cyclohexyl)-ureido]-thiazol-5-ylsulfanyl}-acetic acid or a pharmaceutically acceptable salt thereof.

In another aspect, the present invention relates to methods of treating various disorders by administering a GK activator. In some embodiments, the present invention relates to treating type II diabetes comprising administering to a patient in need thereof a pharmaceutical composition comprising nanoparticles and one or more alkalizers, wherein the nanoparticles have a mean diameter between about 0.5 nm to about 1000 nm, have a polydispersity index of about 0.001 to about 0.400 and comprise a therapeutically effective amount of {2-[3-cyclohexyl-3-(trans-4-propoxy-cyclohexyl)-ureido]-thiazol-5-ylsulfanyl}-acetic acid or a pharmaceutically acceptable salt thereof.

In other embodiments, the present invention relates to a method of improving glycemic control comprising administering to a patient in need thereof a pharmaceutical composition comprising nanoparticles and one or more alkalizers, wherein the nanoparticles have a mean diameter between about 0.5 nm to about 1000 nm, have a polydispersity index of about 0.001 to about 0.400 and comprise a therapeutically effective amount of {2-[3-cyclohexyl-3-(trans-4-propoxy-cyclohexyl)-ureido]-thiazol-5-ylsulfanyl}-acetic acid or a pharmaceutically acceptable salt thereof.

### BRIEF DESCRIPTION OF THE DRAWINGS

FIG. 1. X-Ray Powder Diffraction (XRPD) pattern of nanosized {2-[3-cyclohexyl-3-(trans-4-propoxy-cyclohexyl)-ureido]-thiazol-5-ylsulfanyl}-acetic acid. “A” is the diffraction pattern for lyophilized drug suspension prior to nanosizing. “B” is the diffraction pattern for lyophilized drug suspension after nanosizing.

FIG. 2. Differential scanning calorimetry (DSC) graph of {2-[3-cyclohexyl-3-(trans-4-propoxy-cyclohexyl)-ureido]-thiazol-5-ylsulfanyl}-acetic acid obtained from lyophilizing the drug suspension prior to nanosizing and after nanosizing by microfluidization. “A” is the DSC graph for lyophilized drug suspension prior to nanosizing. “B” is the DSC graph for lyophilized drug suspension after nanosizing.

FIG. 3. XPRD pattern of spray-dried nanoparticles of {2-[3-cyclohexyl-3-(trans-4-propoxy-cyclohexyl)-ureido]-thiazol-5-ylsulfanyl}-acetic acid.

FIG. 4. Dissolution of gelatin capsules of spray dried nanoparticles of {2-[3-cyclohexyl-3-(trans-4-propoxy-cyclohexyl)-ureido]-thiazol-5-ylsulfanyl}-acetic acid and gelatin capsules of nanogranulated {2-[3-cyclohexyl-3-(trans-4-propoxy-cyclohexyl)-ureido]-thiazol-5-ylsulfanyl}-acetic acid.

FIG. 5. In vivo exposure in beagle dogs of i) spray dried nanoparticle capsules containing {2-[3-cyclohexyl-3-(trans-4-propoxy-cyclohexyl)-ureido]-thiazol-5-ylsulfanyl}-acetic acid and ii) capsules of {2-[3-cyclohexyl-3-(trans-4-propoxy-cyclohexyl)-ureido]-thiazol-5-ylsulfanyl}-acetic acid nanogranules, in comparison with iii) {2-[3-cyclohexyl-3-(trans-4-propoxy-cyclohexyl)-ureido]-thiazol-5-ylsulfanyl}-acetic acid in aqueous solution and iv) capsule containing granules formulated with micronized {2-[3-cyclohexyl-3-(trans-4-propoxy-cyclohexyl)-ureido]-thiazol-5-ylsulfanyl}-acetic acid (reference capsule).

FIG. 6. In vivo exposure (AUC) in humans of i) spray dried nanoparticle capsules containing {2-[3-cyclohexyl-3-(trans-4-propoxy-cyclohexyl)-ureido]-thiazol-5-ylsulfanyl}-acetic acid prepared in Example 39 and ii) capsules of {2-[3-cyclohexyl-3-(trans-4-propoxy-cyclohexyl)-ureido]-thiazol-5-ylsulfanyl}-acetic acid nanogranules prepared in Example 40, in comparison a capsule containing granules formulated with micronized {2-[3-cyclohexyl-3-(trans-4-propoxy-cyclohexyl)-ureido]-thiazol-5-ylsulfanyl}-acetic acid (reference capsule).

FIG. 7. In vivo exposure (Cmax) in humans of i) spray dried nanoparticle capsules containing {2-[3-cyclohexyl-3-(trans-4-propoxy-cyclohexyl)-ureido]-thiazol-5-ylsulfanyl}-acetic acid prepared in Example 39 and ii) capsules of {2-[3-cyclohexyl-3-(trans-4-propoxy-cyclohexyl)-ureido]-thiazol-5-ylsulfanyl}-acetic acid nanogranules prepared in Example 40, in comparison a capsule containing granules formulated with micronized {2-[3-cyclohexyl-3-(trans-4-propoxy-cyclohexyl)-ureido]-thiazol-5-ylsulfanyl}-acetic acid (reference capsule).

### DETAILED DESCRIPTION OF THE INVENTION

Novel stable pharmaceutical compositions of {2-[3-cyclohexyl-3-(trans-4-propoxy-cyclohexyl)-ureido]-thiazol-5-ylsulfanyl}-acetic acid or a pharmaceutically acceptable salt thereof, methods of treatment using these pharmaceutical compositions, and methods for preparing these pharmaceutical compositions are provided herein.

{2-[3-cyclohexyl-3-(trans-4-propoxy-cyclohexyl)-ureido]-thiazol-5-ylsulfanyl}-acetic acid is a Class IV drug under the Biopharmaceutics Classification System (BCS), having low solubility and low permeability. As a result, Class IV drugs have poor bioavailability and are usually not well-absorbed while having high variability. Preparing a stable, bioavailable composition containing {2-[3-cyclohexyl-3-(trans-4-propoxy-cyclohexyl)-ureido]-thiazol-5-ylsulfanyl}-acetic acid or a pharmaceutically acceptable salt thereof is, however, not straight forward. Such compositions may exhibit enhanced stability, are highly bioavailable and readily release the active ingredient in the stomach environment, e.g., at pH 1-4, with a desirable dissolution profile.

In one aspect, the present invention provides a pharmaceutical composition comprising solid stabilized particles and a pharmaceutically acceptable excipient, wherein the solid stabilized particles comprise a therapeutically effective amount of {2-[3-cyclohexyl-3-(trans-4-propoxy-cyclohexyl)-ureido]-thiazol-5-ylsulfanyl}-acetic acid or pharmaceutically acceptable salt thereof. In some embodiments, the solid stabilized particles further comprise one or more alkalizers. In other embodiments, the solid stabilized particles further comprise one or more redispersing agents. In yet other embodiments, the solid stabilized particles further comprise one or more redispersing agents and one or more alkalizers. In some embodiments, the present invention relates to a pharmaceutical composition comprising nanoparticles and one or more alkalizers, wherein the nanoparticles have a mean diameter between about 0.5 nm to about 1000 nm, have a polydispersity index of about 0.001 to about 0.400 and comprise {2-[3-cyclohexyl-3-(trans-4-propoxy-cyclohexyl)-ureido]-thiazol-5-ylsulfanyl}-acetic acid or a pharmaceutically acceptable salt thereof. In other embodiments, the present invention relates to a pharmaceutical composition comprising nanoparticles, one or more alkalizers and one or more redispersing agents, wherein the nanoparticles have a mean diameter between about 0.5 nm to about 1000 nm and comprise {2-[3-cyclohexyl-3-(trans-4-propoxy-cyclohexyl)-ureido]-thiazol-5-ylsulfanyl}-acetic acid or a pharmaceutically acceptable salt thereof. The solid stabilized particles comprise {2-[3-cyclohexyl-3-(trans-4-propoxy-cyclohexyl)-ureido]-thiazol-5-ylsulfanyl}-acetic acid or a pharmaceutically acceptable salt thereof in an amount from about 1% to about 80% w/w. In some embodiments, {2-[3-cyclohexyl-3-(trans-4-propoxy-cyclohexyl)-ureido]-thiazol-5-ylsulfanyl}-acetic acid or a pharmaceutically acceptable salt is present in an amount from about 2.5% to about 65% w/w. In other embodiments, {2-[3-cyclohexyl-3-(trans-4-propoxy-cyclohexyl)-ureido]-thiazol-5-ylsulfanyl}-acetic acid or a pharmaceutically acceptable salt is present in an amount from about 5% to about 60% w/w.

Suitable alkalizers include any basic compound that is suitable for oral administration, including, for example, meglumine, sodium carbonate, potassium carbonate, calcium carbonate, magnesium oxide, calcium hydroxide, sodium hydroxide, potassium hydroxide, diethanolamine, potassium bicarbonate, potassium citrate, sodium borate, sodium citrate, triethanolamine, or combinations thereof. Alkalizers can be present in amounts of about 0.1% to about 90% w/w.

In some embodiments, the ratio of alkalizer to active ingredient is between about 2:1 to about 1:50. In other embodiments, the ratio of alkalizer to active ingredient is between about 2:1 to about 1:2. In certain embodiments, the microenvironmental pH of the pharmaceutical composition is more than about 6, for example, more than about 8, more than about 9, more than about 10 or more than about 11. Without wishing to be bound by theory, Applicants believe that microenvironmental pH of the pharmaceutical composition enhances stability of the active agent toward degradation, as well as enhances the dissolution of the active ingredient.

Suitable redispersing agents are agents having good aqueous solubility, are non-hygroscopic and can easily form hydrogen bonds with drug particles. In some embodiments, the redispersing agent is a sugar alcohol. Redispersing agents include, for example, mannitol, trehalose, xylitol, lactose, sucrose, sorbitol, dextran, lactitol, maltitol, erythritol, threitol, arabitol, ribitol, galactitol, fucitol, iditol, inocitol, velomitol, isomalt, inulin or mixtures thereof. Without being bound to any theory, redispersing agents can stabilize microparticles and/or nanoparticles by a mechanism where during the drying process redispersing agent molecules (e.g., sugar alcohols) replace water molecules surrounding the particles, forming hydrogen bonds between redispersing agent molecules and particles, thereby immobilizing particles and limiting particle-particle interaction that leads to aggregation.

### Preparation of Solid Stable Particles

Stable pharmaceutical compositions of the invention comprise stable solid particles. Stable solid particles can be prepared starting with a particle suspension comprising {2-[3-cyclohexyl-3-(trans-4-propoxy-cyclohexyl)-ureido]-thiazol-5-ylsulfanyl}-acetic acid or a pharmaceutically acceptable salt thereof and a solvent, and then removing solvent from the suspension to form stable solid particles. Particle suspensions include microsuspensions, i.e., suspensions comprising particles in the micrometer size range of about 1 μm to about 100 μm, or nanosuspensions, i.e., suspensions comprising particles in the nanometer size range from about 0.5 nm to about 1000 nm, or mixtures thereof. In exemplary embodiments, particle suspensions comprise nanoparticles.

Suitable processes for removing solvent from particle suspensions include for example, granulation, lyophilization, vacuum drying, oven drying, desiccant drying and spray drying. Suitable solvents for particle suspensions include any solvent that is generally recognized as safe (GRAS) by a regulatory authority, e.g., the U.S. Food & Drug Administration, and should be compatible with the drug substance and provide minimal solubility to the drug product. Such solvents include aqueous and organic solvents, for example, water, methanol, heptane, propanol, isopropanol, acetic acid, acetone, ethyl acetate, ethanol, and mixtures thereof. Exemplary particle suspensions comprise water.

Particle suspensions can be prepared by various methods, which can be classified into two categories: top-down methods and bottom-up methods. The top-down method start with bulk materials and break them down to micro- or nano-sized particles by using mechanical, chemical or electrical energy. In certain embodiments, the size reduction method allows for particle size reduction with little or no impact on maintaining crystallinity/polymorphism and stability of a drug substance. Resulting particle suspensions allow for flexibility in formulation. In some embodiments, particle suspensions can be used directly for oral administration, or can be further processed into solid forms (for example by spray drying, granulation or lyphophilization), as well as manufactured into solid dosage forms, for example, tablets and capsules and the like. Oral administration of such formulations described herein can effectively improve drug solubility, reduce dosing amounts, increase dissolution velocity, improve bioavailability, reduce PK variability and alleviate food effects.

Particle suspensions described herein also exhibit chemical stability and show little or no degradation of the drug product into degradation products, even under accelerated conditions.

In top-down methods, larger particles are broken apart to form smaller microsized or nanosized particles. Top-down methods include, for example, microfluidization, wet milling, media milling, rotation-revolution, jet milling, ball milling, micronization or homogenization (e.g., high shear homogenization). Milling methods can utilize various ceramic media, such as ceramic grinding beads (e.g., zirconium milling beads having bead size of about 5 μm to about 500 μm).

Bottom-up methods, on the other hand, synthesize micro- or nano-particle from the atomic or molecular level through chemical reactions or physical processes under strictly selected conditions. Bottom-up methods include, for example, fast evaporation, desolvation, spray drying, lyophilization, precipitation, chemical methods or supercritical fluid processing.

Particle suspensions can comprise one or more stabilizers. In some embodiments, the stabilizers comprise at least one polymeric stabilizer, at least one surfactant stabilizer or a combination thereof. Polymeric stabilizers include, for example, hydroxypropyl cellullose, microcrystalline cellulose, hydroxypropylmethyl cellulose, polyvinyl pyrrolidone, polyvinyl alcohol, polyvinyl sulfate, poloxamer (e.g., poloxamer-188, poloxamer-237, poloxamer-338, poloxamer-407 and other suitable grades of poloxamer can be used), polyethylene glycol, polyethylene glycol-polylactic acid (PEG-PLA), polyethylene oxide, polyoxyethylene alkyl ether, polyoxypropylene glycol alkyl ethers, glucoside alkyl ethers, polyoxyethylene glycol octylphenol ethers, polyoxyethylene glycol alkylphenol ethers, glycerol alkyl ethers, polyoxyethylene glycol sorbitan alkyl esters, sorbitan alkyl esters, cocamide monoethanolamine, cocamide diethanolamine, dodecyldimethylamine oxide, polyethoxylated tallow amine, gelatin, albumin, guar gum, agar or copolymers thereof. In some embodiments, particle suspensions comprise one or more polymeric stabilizers in an amount of about 0.01% w/v to about 40% w/v.

Surfactant stabilizers include, for example, sulfuric acid alkyl ester salts (e.g., sodium lauryl sulfate), dioctyl sulfosuccinate salts (e.g., sodium docusate), sodium deoxycholate, polyoxylene caster oils, polysorbates, polyoxylene stearates, polyoxylglycerides, phospholipids, tocopherol derivatives, bile acid salts, propylene glycol fatty acid mono- or diesters, polyethylene glycol fatty acid esters, polysorbates, polyoxyethylene derivatives of natural oils and waxes, and sorbitan fatty acid esters or mixtures thereof. In other embodiments, particle suspensions comprise from about 0.01% w/v to about 80% w/v surfactant stabilizer.

Particle suspensions described herein have well-controlled and relatively narrow/uniform mean particle size distribution, which can be characterized by measuring polydispersity index (PDI). Polydispersity index is a dimensionless parameter to define the particle size distribution of micro- or nanoparticles obtained from dynamic light scattering analysis. In general, lower PDI values indicate greater particle size uniformity. In some embodiments, particle suspensions comprise particles (i.e., microparticles or nanoparticles) having a PDI of below 0.400. In other embodiments, particle suspensions comprise particles having a PDI of about 0.001 to about 0.400, about 0.001 to about 0.300, 0.001 to about 0.250, about 0.001 to about 0.200, about 0.001 to about 0.190, about 0.001 to about 0.180, about 0.001 to about 0.170, about 0.001 to about 0.160, and about 0.001 to about 0.150.

Particle suspensions can also be characterized by measuring zeta potentials. Zeta potentials reflect the difference in potential between a dispersion medium and stationary layer of fluid attached to the dispersed particle. Zeta potential further indicates the degree of repulsion between adjacent, similarly charged particles in dispersion and is a useful indicator of colloidal stability, i.e., resistance to particle aggregation. In some embodiments, particle suspensions described herein have a zeta potential with an absolute value of greater than 30, for example greater than about 30 mV or less than about −30 mV. In other embodiments, the particle suspensions have a zeta potential of greater than about 50 mV or less than about −50 mV. In yet other embodiments, the particle suspensions have a zeta potential of greater than about 60 mV or less than about −60 mV. In other embodiments, the particle suspensions have a zeta potential of greater than about 80 mV or less than about −80 mV. In other embodiments, the particle suspensions have a zeta potential of greater than about 100 mV or less than about −100 mV. In yet other embodiments, the particle suspensions have a zeta potential of between about −30 mV and −100 mV or between about 30 mV to about 100 mV.

In some embodiments, particle suspensions described herein have a solid concentration of about 0.5% to about 80% w/v. In other embodiments, particle suspensions have a viscosity of about 0.5 cps to about 600 cps.

Top-down and bottom-up techniques can form drug microparticles and/or nanoparticles in suspension having uncompromised physical stability, e.g., little or negligible particle size change over time, little or negligible degradation of the drug product, little or negligible change in crystallinity/polymorphism. However, challenges arise when converting particle suspensions into solid forms where it is difficult to maintain stability, such as avoiding particle aggregation while maintaining particle size, and avoiding interconversion among polymorph forms. Stable solid microparticles and/or nanoparticles are provided by admixing one or more redispersing agents with a particle suspension prior to converting the particle suspension into a solid form.

In some embodiments, particle suspensions comprise one or more redispersing agents in an amount of about 0.1% to about 90% w/w. In other embodiments, redispersing agents can be present from about 1% to about 80% w/w or about 5% to about 70% w/w. In some embodiments, redispersing agents can be present from about 2.5% to about 10% w/w. In other embodiments, redispersing agents can be present from about 25% to about 40% w/w.

Solid stabilized particles can be formed from the particle suspensions described herein using various known methods including, for example, spray drying, wet granulation, dry granulation, steam granulation techniques, melt granulation techniques, moisture-activated dry granulation techniques (MADG), moist granulation techniques (MGT), thermal adhesion granulation processes (TAGP), foam granulation techniques, lyophilization, vacuum drying, oven drying, desiccant drying and the like.

In some embodiments, the mean solid particle size is between about 1 μm to about 100 μm. In other embodiments, the mean solid particle size is between about 2 μm to about 90 μm. In yet other embodiments, the mean solid particle size is between about 5 μm to about 80 μm. In other embodiments, the mean solid particle size is between about 10 μm and about 70 μm.

In further embodiments, the mean solid particle size is between about 0.5 nm to about 1000 nm. In other embodiments, the mean solid nanoparticle size is less than about 900 nm. In other embodiments, the mean solid nanoparticle size is between about 0.5 nm to about 800 nm. In yet other embodiments, the mean solid nanoparticle size is between about 200 nm to about 400 nm.

Solid stabilized particles formed from the particle suspensions described herein also have well-controlled and relatively narrow mean particle size distribution. Without being bound to any theory, narrow mean particle size distribution can provide highly bioavailable pharmaceutical compositions having consistent drug delivery with less PK variability. In some embodiments, solid stabilized particles formed from the particle suspensions described herein have a polydispersity index (PDI) of below 0.400. In other embodiments, solid stabilized particles formed from the particle suspensions described herein have a PDI of about 0.001 to about 0.400, 0.001 to about 0.300, 0.001 to about 0.250, about 0.001 to about 0.200, about 0.001 to about 0.190, about 0.001 to about 0.180, about 0.001 to about 0.170, about 0.001 to about 0.160, and about 0.001 to about 0.150.

In some embodiments, spray drying is used to make solid stabilized particles comprising {2-[3-cyclohexyl-3-(trans-4-propoxy-cyclohexyl)-ureido]-thiazol-5-ylsulfanyl}-acetic acid or a pharmaceutically acceptable salt thereof. In other embodiments, spray dried solid stabilized particles can be used directly as a solid dosage form or further formulated into a solid dosage form, such as a tablet and the like.

In other embodiments, wet granulation is used to make solid stabilized particles comprising {2-[3-cyclohexyl-3-(trans-4-propoxy-cyclohexyl)-ureido]-thiazol-5-ylsulfanyl}-acetic acid or a pharmaceutically acceptable salt thereof. Wet granulation methods include, for example, standard wet granulation techniques and specialized wet granulation techniques, such as high-shear mixture granulation, fluid-bed granulation, extrusion, spheronization lyophilization, spray dry granulation and the like. In certain embodiments, fluid-bed granulation or spray dry granulation is used to make solid stabilized particles comprising {2-[3-cyclohexyl-3-(trans-4-propoxy-cyclohexyl)-ureido]-thiazol-5-ylsulfanyl}-acetic acid or a pharmaceutically acceptable salt thereof. Dry granulation methods include standard dry granulation and specialized dry granulation techniques, such as slugging, roller compaction, and the like. Melt granulation methods include thermoplastic melt granulation and the like.

Wet granulation methods involve the use of a liquid binder solution comprising one or more binders. Liquid binder solutions can be mixed with a powder to cause the powder to agglomerate lightly, thereby forming granules. In some embodiments, one or more redispersing agents are added to the liquid binder solution, which is then added to a particle suspension of the present invention prior to granulation. Following granule formation, the granules are typically dried and sized (using, e.g., mesh screens). In some embodiments, the granules can be milled to achieve a desired particle size. Both low-shear and high-shear mixing equipment can be utilized.

Suitable binders include cellulose derivatives (e.g., hydroxypropylmethyl cellulose, hydroxypropylmethyl cellulose acetate, hydroxypropylmethyl cellulose phthalate, hydroxypropyl cellulose, methylcellulose, hydroxyethyl cellulose, hydroxethyl cellulose acetate, and the like), monosaccharides (e.g., dextrose and the like), polysaccharides/oligosaccharides (e.g., dextrin, maltodextrin, pectin, maltose, polydextrose, starch and the like), polyvinyl pyrrolidone, polyvinyl alcohol, polyvinyl caprolactam, carbomer, povidone, copovidone, gelatin, natural gums (e.g., guar gum, acacia, carrageenan, agar, alginic acid, gum arabic, and the like), poloxamer, polycarbophil or mixtures thereof. Binders can be present in amounts from about 0.01% to about 20% w/w dry weight.

Binder solutions can also include one or more fillers, diluents, disintegrants or mixtures thereof. Suitable filler/diluents include, for example, microcrystalline cellulose, dicalcium phosphate, lactose, starch, calcium carbonate, calcium lactate, calcium phosphate, calcium silicate, calcium sulfate, hypromellose, pregelatinzed starch, dextrin, magnesium carbonate, magnesium oxide, maltodextrin, maltose, polydextrose, polymethacrylate, simethicone, sodium alginate, sodium carbonate, mannitol, trehalose, xylitol, lactose, sucrose, sorbitol, lactitol, maltitol, erythritol, threitol, arabitol, ribitol, galactitol, fucitol, iditol, inocitol, velomitol, isomalt or mixtures thereof. Fillers/diluents can be present in amounts from about 0.1% to about 99% w/w dry weight.

Suitable disintegrants include, for example, croscarmellose sodium, sodium starch glycolate, microcrystal cellulose, crospovidone, pregelatinized starch, sodium alginate, chitosan, magnesium aluminum silicate; methyl cellulose, guar gum or mixtures thereof. Disintegrants can be present in amounts from about 0.01% to about 30% w/w dry weight.

### Dosage Forms

The invention further provides pharmaceutical compositions in forms for oral administration. Such pharmaceutical compositions exhibit chemical stability and show little or no degradation of the drug product into degradation products. Pharmaceutical compositions can be in solid or liquid form. In one embodiment, the pharmaceutical composition is a solid composition. Pharmaceutical compositions comprise solid stabilized particles described herein.

In one aspect, pharmaceutical compositions of the present invention may be prepared by controlling microenvironmental pH of the composition. Thus, in one embodiment, the present invention relates to pharmaceutical compositions (e.g., solid oral dosage forms) comprising solid stabilized particles and a compound that modulates the pH environment of the composition (e.g., an alkalizer), wherein the solid stabilized particles comprise a therapeutically effective amount of {2-[3-cyclohexyl-3-(trans-4-propoxy-cyclohexyl)-ureido]-thiazol-5-ylsulfanyl}-acetic acid or a pharmaceutically acceptable salt thereof.

In some embodiments, pharmaceutical compositions comprise about 0.5 mg to about 1200 mg of {2-[3-cyclohexyl-3-(trans-4-propoxy-cyclohexyl)-ureido]-thiazol-5-ylsulfanyl}-acetic acid or a pharmaceutically acceptable salt thereof. In other embodiments, pharmaceutical compositions comprise about 50 mg, about 100 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, about 1000 mg, about 1050 mg, about 1100 mg, about 1150 mg or about 1200 mg of {2-[3-cyclohexyl-3-(trans-4-propoxy-cyclohexyl)-ureido]-thiazol-5-ylsulfanyl}-acetic acid or a pharmaceutically acceptable salt thereof.

Solid oral compositions of the present invention can be formulated to have an immediate release profile as referenced by FDA guidelines (“Dissolution Testing of Immediate Release Solid Oral Dosage Forms”, issued August 1997, Section IV-A). In the dissolution testing guideline for immediate release profiles, materials which dissolve at least 80% in the first 30 to 60 minutes in solution qualify as immediate release profiles. Therefore, in one embodiment, solid oral compositions release of most or all the active ingredient over a short period of time, such as 60 minutes or less, and make rapid absorption of the drug possible. In other embodiments, solid oral compositions release about 80% of the drug over about 15 minutes.

In some embodiments of the invention, the solid composition further comprises at least one additional pharmaceutical ingredient. Additional pharmaceutical ingredients include any component or excipient other than powdered pharmaceutically acceptable carriers, so long as the material is not generally deleterious to a human subject when the solid composition is administered at dosing quantities. Non-limiting examples of additional ingredients include: glidants and lubricants, such as colloidal silica, talc, magnesium stearate, calcium stearate, stearic acid, solid polyethylene glycol, sodium oleate, sodium stearate, sodium benzoate, sodium acetate, sodium chloride, sodium stearyl furamate, and sodium lauryl sulfate; solubilizing agents, such as agar-agar, calcium carbonate, sodium carbonate, croscarmellose sodium, starches, pregelatinized starches, sodium starch glycolate, crospovidone, methyl cellulose, agar, bentonite, xanthan gum, alginic acid, and certain silicates; solution retarding agents, such as polymers, for example biodegradable polymers such as polylactic acid, polyepsilon caprolactone, polyhydroxy butyric acid, polyorthoesters, polyacetals, polydihydropyrans, polycyanoacrylates, and cross-linked or amphipathic block copolymers of hydrogelsparaffin, and wax, for example, paraffin; resorption accelerating agents, such as quaternary ammonium compounds; absorption agents, such as quaternary ammonium compounds, bentonite, kaolin, or dicalcium phosphate.

The pharmaceutical compositions of the invention can be prepared by various means. Such compositions comprise solid stabilized particles. In some embodiments, the solid stabilized particles are provided as powder.

In some embodiments, capsules may be prepared by, for example, obtaining solid stabilized particles described herein containing {2-[3-cyclohexyl-3-(trans-4-propoxy-cyclohexyl)-ureido]-thiazol-5-ylsulfanyl}-acetic acid or a pharmaceutically acceptable salt thereof and encapsulating the solid stabilized particles with gelatin or another suitable shell material. In some embodiments, the solid stabilized particles are provided as a powder. Additional ingredients, such as those described herein, including alkalizers, binders, fillers, diluents, glidants, lubricants, disintegrating agents, solubilizing agents, or mixtures thereof may be combined with the solid stabilized particles prior to encapsulation.

In other embodiments, tablets may be prepared by, for example, obtaining solid stabilized particles described herein containing {2-[3-cyclohexyl-3-(trans-4-propoxy-cyclohexyl)-ureido]-thiazol-5-ylsulfanyl}-acetic acid or a pharmaceutically acceptable salt thereof and pressing the solid stabilized particles into tablets using conventional methods. In some embodiments, the solid stabilized particles are provided as a powder. Additional ingredients, such as those described herein, including binders, fillers, diluents, glidants, lubricants, disintegrating agents, solubilizing agents, solution retardants, absorption agents, or mixtures thereof, may be added to the solid stabilized particles before pressing into tablets.

The tablets described herein may be either uncoated or coated. In various embodiments, tablets are coated with a clear or opaque protective coating, which may for example, comprise a sealing coat of shellac, a coating of sugar or polymeric material, and/or a polish coating of wax. In various embodiments, tablets are coated to delay disintegration and absorption in the gastrointestinal tract and thereby provide a sustained action over a longer period. Such coatings may comprise glyceryl monostearate or glyceryl distearate. Additionally, dyestuffs can be added to these coatings to distinguish different unit dosages.

The dosage of the pharmaceutical composition of the present invention will vary depending on the symptoms, the treatment desired, age and body weight of the subject, the nature and severity of the disorder to be treated, the route of administration and pharmacokinetics of the active ingredients. The frequency of the dose indicated will also vary with the treatment desired and the disorder indicated.

In one embodiment, {2-[3-cyclohexyl-3-(trans-4-propoxy-cyclohexyl)-ureido]-thiazol-5-ylsulfanyl}-acetic acid or a pharmaceutically acceptable salt is administered in an amount sufficient to achieve a therapeutic effect. The dosage range for {2-[3-cyclohexyl-3-(trans-4-propoxy-cyclohexyl)-ureido]-thiazol-5-ylsulfanyl}-acetic acid or a pharmaceutically acceptable salt can be from about 0.5 mg to about 2400 mg per day in one or more administrations. In other embodiments, {2-[3-cyclohexyl-3-(trans-4-propoxy-cyclohexyl)-ureido]-thiazol-5-ylsulfanyl}-acetic acid or a pharmaceutically acceptable salt can be administered in amounts from about 5 mg to about 1200 mg per day, or about 10 mg to about 800 mg per day in one or more administrations. In some embodiments, {2-[3-cyclohexyl-3-(trans-4-propoxy-cyclohexyl)-ureido]-thiazol-5-ylsulfanyl}-acetic acid or a pharmaceutically acceptable salt can be administered in one or more administrations for a total daily amount of about 100 mg, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, about 1000 mg, about 1100 mg, about 1200 mg, about 1300 mg, about 1400 mg, about 1500 mg, about 1600 mg, about 1700 mg, about 1800 mg, about 1900 mg, about 2000 mg, about 2100 mg, about 2200 mg, about 2300 mg or about 2400 mg.

In some embodiments, the dosage of {2-[3-cyclohexyl-3-(trans-4-propoxy-cyclohexyl)-ureido]-thiazol-5-ylsulfanyl}-acetic acid or a pharmaceutically acceptable salt can be in an amount from about 0.001 mg/kg of body weight per day to about 100 mg/kg of body weight per day. In other embodiments, the dosage of {2-[3-cyclohexyl-3-(trans-4-propoxy-cyclohexyl)-ureido]-thiazol-5-ylsulfanyl}-acetic acid or a pharmaceutically acceptable salt can be in an amount from about 0.003 mg/kg of body weight per day to about 60 mg/kg of body weight per day. In yet other embodiments, the dosage of {2-[3-cyclohexyl-3-(trans-4-propoxy-cyclohexyl)-ureido]-thiazol-5-ylsulfanyl}-acetic acid or a pharmaceutically acceptable salt can be in an amount of about 0.5 mg/kg of body weight per day, about 1 mg/kg of body weight per day, about 2 mg/kg of body weight per day, about 5 mg/kg of body weight per day, about 10 mg/kg of body weight per day, about 20 mg/kg of body weight per day, about 40 mg/kg of body weight per day or about 60 mg/kg of body weight per day. One skilled in the art will appreciate that the administered doses can be converted to suitable human equivalent doses.

Pharmaceutical compositions described herein may exhibit improved biovailability of {2-[3-cyclohexyl-3-(trans-4-propoxy-cyclohexyl)-ureido]-thiazol-5-ylsulfanyl}-acetic acid or a pharmaceutically acceptable salt thereof upon administration to a subject relative to reference compositions that do not include a solid stabilized particle described herein.

### Methods of Use:

The present invention also provides methods for treating a disease, disorder or condition that can be managed by activating glucokinase in a subject (e.g., a mammal, such as a human) by administering to a patient in need thereof a stable pharmaceutical composition described herein. Such methods including, for example, treating type I diabetes and/or type II diabetes; normalizing or lowering blood glucose levels; improving glucose tolerance; improving glycemic control; reducing fasting plasma glucose; reducing postprandial plasma glucose; reducing glycosylated hemoglobin HbA1c; slowing progression of, delaying or treating complications of diabetes, e.g., diabetic nephropathy, retinopathy, neuropathy or cardiovascular disease; reducing weight or preventing an increase of weight or facilitating a reduction of weight; treating the degeneration of pancreatic beta cells; improving and/or restoring functionality of pancreatic beta cells; stimulating and/or restoring functionality of pancreatic insulin secretion; enhancing phosphorylation of glucose; or maintaining and/or improving insulin sensitivity; and/or treating or preventing hyperinsulinemia and/or insulin resistance.

In one embodiment, the present invention relates to treating type II diabetes comprising administering to a patient in need thereof a pharmaceutical composition comprising nanoparticles and one or more alkalizers, wherein the nanoparticles have a mean diameter between about 0.5 nm to about 1000 nm, have a polydispersity index of about 0.001 to about 0.400 and comprise a therapeutically effective amount of {2-[3-cyclohexyl-3-(trans-4-propoxy-cyclohexyl)-ureido]-thiazol-5-ylsulfanyl}-acetic acid or a pharmaceutically acceptable salt thereof. In other embodiments, the nanoparticles further comprise one or more redispersing agents.

In another embodiment, the present invention provides for normalizing blood glucose levels and improving glucose tolerance by administering to a patient in need thereof a pharmaceutical composition comprising nanoparticles and one or more alkalizers, wherein the nanoparticles have a mean diameter between about 0.5 nm to about 1000 nm, have a polydispersity index of about 0.001 to about 0.400 and comprise a therapeutically effective amount of {2-[3-cyclohexyl-3-(trans-4-propoxy-cyclohexyl)-ureido]-thiazol-5-ylsulfanyl}-acetic acid or a pharmaceutically acceptable salt thereof. In other embodiments, the nanoparticles further comprise one or more redispersing agents.

In another embodiment, the present invention provides for improving glycemic control; and/or for reducing fasting plasma glucose, reducing postprandial plasma glucose and/or reducing glycosylated hemoglobin HbA1c by administering to a patient in need thereof a pharmaceutical composition comprising nanoparticles and one or more alkalizers, wherein the nanoparticles have a mean diameter between about 0.5 nm to about 1000 nm, have a polydispersity index of about 0.001 to about 0.400 and comprise a therapeutically effective amount of {2-[3-cyclohexyl-3-(trans-4-propoxy-cyclohexyl)-ureido]-thiazol-5-ylsulfanyl}-acetic acid or a pharmaceutically acceptable salt thereof. In other embodiments, the nanoparticles further comprise one or more redispersing agents.

In some embodiments, the administration of {2-[3-cyclohexyl-3-(trans-4-propoxy-cyclohexyl)-ureido]-thiazol-5-ylsulfanyl}-acetic acid or a pharmaceutically acceptable salt thereof may reduce the levels of HbA1C in a subject in need thereof. In other embodiments, the administration of {2-[3-cyclohexyl-3-(trans-4-propoxy-cyclohexyl)-ureido]-thiazol-5-ylsulfanyl}-acetic acid or a pharmaceutically acceptable salt thereof may reduce the amount of HbA1C in a subject in need thereof by at least 0.1 of a percentage point, or 0.2 of a percentage point, or 0.3 of a percentage point, or 0.4 of a percentage point, or 0.5 of a percentage point, or 0.6 of a percentage point, or 0.7 of a percentage point, or 0.8 of a percentage point, or 0.9 of a percentage point, or one percentage point. In still other embodiments, the administration of {2-[3-cyclohexyl-3-(trans-4-propoxy-cyclohexyl)-ureido]-thiazol-5-ylsulfanyl}-acetic acid or a pharmaceutically acceptable salt thereof may reduce the level of HbA1C in a subject in need thereof to less than 7%. In other embodiments, the level of HbA1C may be reduced to a level between 5 and 6.5%.

In another embodiment, the present invention provides for slowing progression of, delaying or treating complications (e.g., diabetic nephropathy, retinopathy, neuropathy or cardiovascular disease) by administering to a patient in need thereof a pharmaceutical composition comprising nanoparticles and one or more alkalizers, wherein the nanoparticles have a mean diameter between about 0.5 nm to about 1000 nm, have a polydispersity index of about 0.001 to about 0.400 and comprise a therapeutically effective amount of {2-[3-cyclohexyl-3-(trans-4-propoxy-cyclohexyl)-ureido]-thiazol-5-ylsulfanyl}-acetic acid or a pharmaceutically acceptable salt thereof. In other embodiments, the nanoparticles further comprise one or more redispersing agents.

In yet another embodiment, the present invention provides for reducing weight or preventing an increase of weight or facilitating a reduction of weight by administering to a patient in need thereof a pharmaceutical composition comprising nanoparticles and one or more alkalizers, wherein the nanoparticles have a mean diameter between about 0.5 nm to about 1000 nm, have a polydispersity index of about 0.001 to about 0.400 and comprise a therapeutically effective amount of {2-[3-cyclohexyl-3-(trans-4-propoxy-cyclohexyl)-ureido]-thiazol-5-ylsulfanyl}-acetic acid or a pharmaceutically acceptable salt thereof. In other embodiments, the nanoparticles further comprise one or more redispersing agents.

In another embodiment, the present invention provides for treating the degeneration of pancreatic beta cells; and/or improving and/or restoring functionality of pancreatic beta cells; and/or stimulating and/or restoring functionality of pancreatic insulin secretion by administering to a patient in need thereof a pharmaceutical composition comprising nanoparticles and one or more alkalizers, wherein the nanoparticles have a mean diameter between about 0.5 nm to about 1000 nm, have a polydispersity index of about 0.001 to about 0.400 and comprise a therapeutically effective amount of {2-[3-cyclohexyl-3-(trans-4-propoxy-cyclohexyl)-ureido]-thiazol-5-ylsulfanyl}-acetic acid or a pharmaceutically acceptable salt thereof. In other embodiments, the nanoparticles further comprise one or more redispersing agents.

In another embodiment, the present invention provides for maintaining and/or improving insulin sensitivity; and/or treating or preventing hyperinsulinemia and/or insulin resistance by administering to a patient in need thereof a pharmaceutical composition comprising nanoparticles and one or more alkalizers, wherein the nanoparticles have a mean diameter between about 0.5 nm to about 1000 nm, have a polydispersity index of about 0.001 to about 0.400 and comprise a therapeutically effective amount of {2-[3-cyclohexyl-3-(trans-4-propoxy-cyclohexyl)-ureido]-thiazol-5-ylsulfanyl}-acetic acid or a pharmaceutically acceptable salt thereof. In other embodiments, the nanoparticles further comprise one or more redispersing agents.

In yet another embodiment, the present invention provides for decreasing the daily dose of insulin by administering to a patient in need thereof a pharmaceutical composition comprising nanoparticles and one or more alkalizers, wherein the nanoparticles have a mean diameter between about 0.5 nm to about 1000 nm, have a polydispersity index of about 0.001 to about 0.400 and comprise a therapeutically effective amount of {2-[3-cyclohexyl-3-(trans-4-propoxy-cyclohexyl)-ureido]-thiazol-5-ylsulfanyl}-acetic acid or a pharmaceutically acceptable salt thereof. In other embodiments, the nanoparticles further comprise one or more redispersing agents.

In yet another embodiment, the present invention provides for treating a condition in a subject comprising administering to a patient in need thereof a pharmaceutical composition comprising nanoparticles and one or more alkalizers, wherein the nanoparticles have a mean diameter between about 0.5 nm to about 1000 nm, have a polydispersity index of about 0.001 to about 0.400 and comprise a therapeutically effective amount of {2-[3-cyclohexyl-3-(trans-4-propoxy-cyclohexyl)-ureido]-thiazol-5-ylsulfanyl}-acetic acid or a pharmaceutically acceptable salt thereof, wherein the condition is selected from metabolic disorders (including metabolic syndrome), glucose intolerance, prediabetic state, insulin resistance, blood glucose lowering, hyperglycemia, impaired glucose tolerance (IGT), Syndrome X, impaired fasting glucose (IFG), type II diabetes, type I diabetes, delaying IGT to type II diabetes, delaying the progression of non-insulin-requiring type II diabetes to insulin-requiring type II diabetes, dyslipidemia, hyperlipidemia, hyperlipoproteinemia, hypertension, osteoporosis, non-alcoholic fatty liver disease (NAFLD), complications resulting from or associated with diabetes, (including nephropathy, retinopathy, neuropathy, impaired wound healing)) cardiovascular disease (including arteriosclerosis, atherosclerosis), lowering of food intake, appetite regulation, obesity, regulating feeding behavior, and enhancing secretion of enteroincretins. In other embodiments, the nanoparticles further comprise one or more redispersing agents.

In other embodiments, the present invention provides for methods of treatment described herein as an adjunct to diet and exercise in subjects with type II diabetes or type I diabetes.

### DEFINITIONS

The term “pharmaceutically acceptable” means biologically or pharmacologically compatible for in vivo use in animals or humans, and preferably means approved by a regulatory agency of the Federal or a state government or listed in the U.S. Pharmacopeia or other generally recognized pharmacopeia for use in animals, and more particularly in humans.

The term “treating,” “treatment,” and “treat” refer to managing or controlling a disease, condition or disorder. This includes relieving, alleviating, ameliorating, delaying, reducing, reversing, improving a disease, disorder or condition or at least one symptom thereof, depending on the nature of the disease, disorder, or condition and its characteristic symptoms.

The term “subject” means animals. The subject can be any animal in the context of a trial or screening or activity experiment. Thus, as can be readily appreciated by one of ordinary skill in the art, the methods, compounds, and formulations of the present invention are particularly suited to administration to any animal, particularly a mammal, and including, but not limited to, humans, domestic animals, such as feline or canine subjects, farm animals, such as bovine, equine, caprine, ovine and porcine subjects, wild animals, research animals, such as mice, rats, rabbits, goats, sheep, pigs, dogs, cats etc., avian species for veterinary medical use.

The terms “effective amount” and “therapeutically effective” refer to an amount or quantity of a compound or pharmaceutical formulation that is sufficient to result in a desired biological or therapeutic response in a tissue, system, or subject in need thereof. For example, the terms “effective amount” and “therapeutically effective amount” refer to an amount of {2-[3-cyclohexyl-3-(trans-4-propoxy-cyclohexyl)-ureido]-thiazol-5-ylsulfanyl}-acetic acid or a pharmaceutically acceptable salt thereof that is sufficient to produce an effective response upon administration to a subject. The “therapeutically effective amount” will vary depending on the compound, the disease and its severity and the age, weight, physical condition and responsiveness of the subject to be treated.

The term “about” or “approximately” means within an acceptable error range for the particular value as determined by one of ordinary skill in the art, which will depend in part on how the value is measured or determined, i.e., the limitations of the measurement system. For example, “about” can mean within 1 or more than 1 standard deviation, per practice in the art. Alternatively, “about” with respect to the formulations can mean plus or minus a range of up to 30%, up to 20%, up to 10% and even up to 5%.

The pharmacokinetic parameters described herein include area under the plasma concentration-time curve (AUC₀₋ₜ and AUC_{0-∞}), maximum plasma concentration (Cₘₐₓ) and time of maximum plasma concentration (Tₘₐₓ). The time of maximum concentration, Tₘₐₓ, is determined as the time corresponding to Cₘₐₓ. Area under the plasma concentration-time curve up to the time corresponding to the last measurable concentration (AUC₀₋ₜ) is calculated by numerical integration using the linear trapezoidal rule as follows:

$${AUC}_{0 - t} = {\sum }_{i - 2} 0.5 \cdot ( {C}_{i} = {C}_{i - 1} ) \cdot ( {t}_{i} - {t}_{i - 1} ) \qquad Eq . 1$$

where Cᵢ is the plasma concentrations of {2-[3-cyclohexyl-3-(trans-4-propoxy-cyclohexyl)-ureido]-thiazol-5-ylsulfanyl}-acetic acid at the corresponding sampling time point tᵢ and n is the number of time points up to and including the last quantifiable concentration.

The area under the plasma concentration-time curve from time zero to infinity is calculated according to the following equation:

$${AUC}_{0 - ∞} = {AUC}_{0 - t} + \frac{{C}_{last}}{{λ}_{z}} \qquad Eq . 2$$

where Cₗₐₛₜ is the last measurable concentration.

The following examples are merely illustrative of the present invention and should not be construed as limiting the scope of the invention in any way. Many variations and equivalents that are encompassed by the present invention will become readily apparent to those skilled in the art upon reading the present disclosure.

### EXAMPLES

### A. Preparation of Particle Suspensions

### General Experimental Procedure to Prepare Microsuspensions or Nanosuspensions of {2-[3-cyclohexyl-3-(trans-4-propoxy-cyclohexyl)-ureido]-thiazol-5-ylsulfanyl}-acetic acid (drug ingredient)

A polymeric stabilizer (10 g, 1% w/v) was added to 1 L purified water with mixing until a clear solution was obtained. A surfactant stabilizer (5 g, 0.5% w/v) was added to the solution with mixing until a clear solution was obtained. {2-[3-cyclohexyl-3-(trans-4-propoxy-cyclohexyl)-ureido]-thiazol-5-ylsulfanyl}-acetic acid (100 g, 10% w/v) was then added stepwise with mixing until a uniform suspension was obtained. The suspension was microfluidized using a Microfluidizer M-110EH equipped with a mixing chamber of 200 microns and interaction of 80 microns at a mill pressure of between about 20,000 to about 30,000 psi until there was no further particle size reduction. The resulting particle suspension was collected.

### Example 1

A nanosuspension of {2-[3-cyclohexyl-3-(trans-4-propoxy-cyclohexyl)-ureido]-thiazol-5-ylsulfanyl}-acetic acid was prepared using the method above, where the polymeric stabilizer was hydroxypropyl methylcellulose (HPMC) and the surfactant stabilizer was sodium lauryl sulfate (SLS). The resulting nanosuspension had a 10% solid content, a mean particle size of 225.6 nm, a polydispersity index of 0.145 and a zeta potential of −57.6 mV.

The physical stability of the nanosuspension is shown in the Table 1 below, where no agglomeration was observed after storage at room temperature for 6-48 hours and at 5° C. for 1.5 months.

TABLE 1
Physical stability of nanoparticle suspension

| Time/Temp | Mean particle size (nm) |
|---|---|
| 0 | 225.6 |
| 6 h, RT | 223.1 |
| 24 h, RT | 230.9 |
| 48 h, RT | 229.4 |
| 1.5 month, 5° C. | 226.0 |

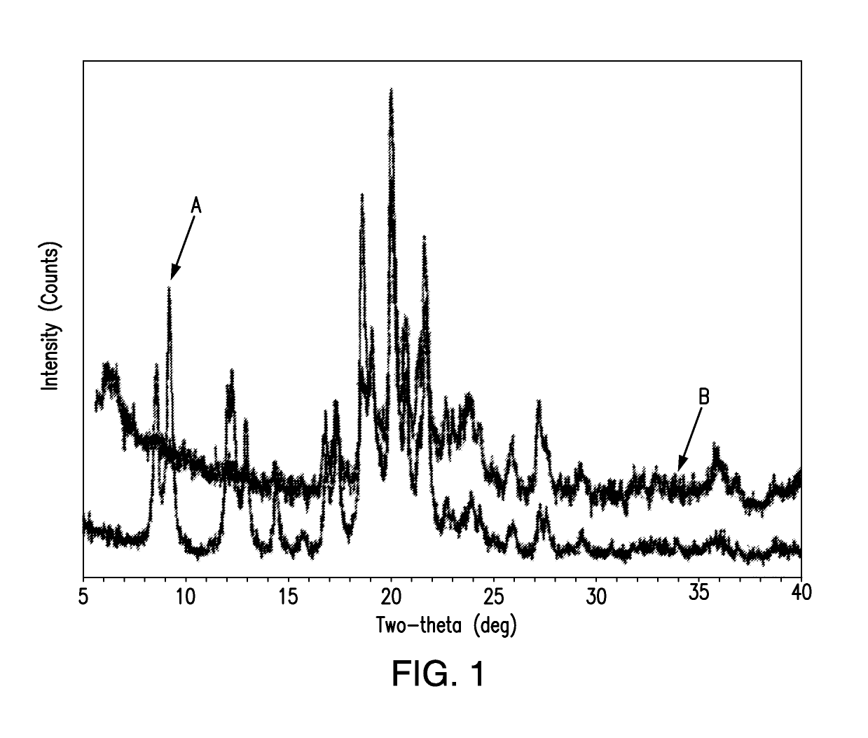

FIG. 1 shows X-ray powder diffraction (XRPD) patterns of {2-[3-cyclohexyl-3-(trans-4-propoxy-cyclohexyl)-ureido]-thiazol-5-ylsulfanyl}-acetic acid obtained from lyophilizing the drug suspension prior to nanosizing (“A”) and after nanosizing (“B”) by microfluidization. The crystal structure of {2-[3-cyclohexyl-3-(trans-4-propoxy-cyclohexyl)-ureido]-thiazol-5-ylsulfanyl}-acetic acid was not changed during nanosizing.

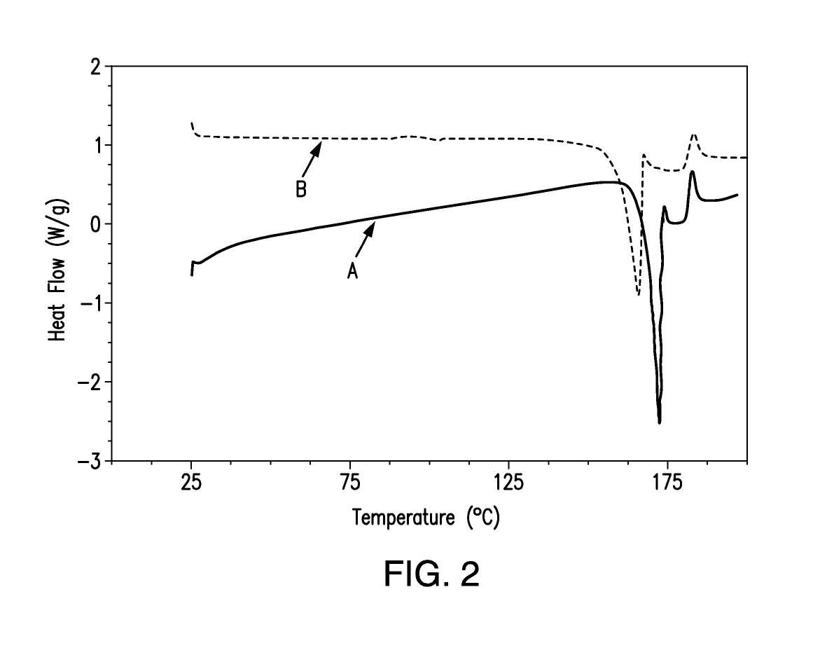

FIG. 2 shows a differential scanning calorimetry graph of {2-[3-cyclohexyl-3-(trans-4-propoxy-cyclohexyl)-ureido]-thiazol-5-ylsulfanyl}-acetic acid obtained from lyophilizing the drug suspension prior to nanosizing (“A”) and after nanosizing (“B”) by microfluidization. Chemical stability of the drug suspension before and after nanosizing (via monitoring drug degradation induced by the nanosizing process) is shown in Table 2 below.

TABLE 2
Degradation profile of active ingredient suspension before and after nanosizing

| | Degradant profile | | | | | | | | | |
|---|---|---|---|---|---|---|---|---|---|---|
| | | | | | | | | | | Drug |
| | | | | | | | | | | Ingredient |
| Sample | A | B | C | D | E | F | G | H | I | % Assay |
| Suspension | 0.04 | 0.02 | 0.04 | 0.2 | 0.05 | 0.04 | 0.02 | 0.02 | 0.02 | 99.49 |
| before | | | | | | | | | | |
| nanosizing | | | | | | | | | | |
| 100 μg/mL | | | | | | | | | | |
| Nanosuspension | 0.04 | 0.02 | 0.04 | 0.19 | 0.05 | 0.04 | 0.04 | 0.02 | 0.02 | 99.47 |
| 100 μg/mL | | | | | | | | | | |

Chemical stability of the nanosuspension stored under refrigerated conditions is shown in Table 3 below.

TABLE 3
Degradation profile of drug ingredient nanosuspension stored at 5° C.

| | Degradant profile | | | | | | | | | |
|---|---|---|---|---|---|---|---|---|---|---|
| | | | | | | | | | | Drug |
| | | | | | | | | | | Ingredient |
| Sample | A | B | C | D | E | F | G | H | I | % Assay |
| 3 wks T-1 | 0.09 | 0.03 | 0.03 | 0.2 | 0.04 | 0.03 | 0.02 | 0.01 | 0.02 | 99.53 |
| 3 wks T-2 | 0.07 | 0 | 0.03 | 0.19 | 0.04 | 0.04 | 0.03 | 0.01 | 0.02 | 99.57 |
| 2 month T-1 | 0.07 | 0 | 0.04 | 0.19 | 0.04 | 0.03 | 0.03 | 0.02 | 0.02 | 99.56 |
| 2 month T-2 | 0.07 | 0 | 0.04 | 0.19 | 0.04 | 0.03 | 0.02 | 0.02 | 0.02 | 99.59 |

### Example 2

A nanosuspension of {2-[3-cyclohexyl-3-(trans-4-propoxy-cyclohexyl)-ureido]-thiazol-5-ylsulfanyl}-acetic acid was prepared using the method above, where the polymeric stabilizer was hydroxypropyl cellulose (HPC) and the surfactant stabilizer was sodium lauryl sulfate (SLS). The resulting nanosuspension had a 10% solid content, a mean particle size of 252.2 nm, a polydispersity index of 0.171 and a zeta potential of −55.6 mV.

### Example 3

A nanosuspension of {2-[3-cyclohexyl-3-(trans-4-propoxy-cyclohexyl)-ureido]-thiazol-5-ylsulfanyl}-acetic acid was prepared using the method above, where the polymeric stabilizer was poloxamer 188 and the surfactant stabilizer was sodium lauryl sulfate (SLS). The resulting nanosuspension had a 10% solid content, a mean particle size of 260.4 nm, a polydispersity index of 0.183 and a zeta potential of −54.4 mV.

### Example 4

A nanosuspension of {2-[3-cyclohexyl-3-(trans-4-propoxy-cyclohexyl)-ureido]-thiazol-5-ylsulfanyl}-acetic acid was prepared using the method above, where the polymeric stabilizer was polyvinyl alcohol and the surfactant stabilizer was sodium lauryl sulfate (SLS). The resulting nanosuspension had a 10% solid content, a mean particle size of 261.4 nm, a polydispersity index of 0.166 and a zeta potential of −57.3 mV.

### Example 5

A nanosuspension of {2-[3-cyclohexyl-3-(trans-4-propoxy-cyclohexyl)-ureido]-thiazol-5-ylsulfanyl}-acetic acid was prepared using the method above, where the polymeric stabilizer was hydroxypropyl cellulose and the surfactant stabilizer was sodium lauryl sulfate (SLS). The resulting nanosuspension had a 10% solid content, a mean particle size of 252.2 nm, a polydispersity index of 0.171 and a zeta potential of −55.6 mV.

### Example 6

A nanosuspension of {2-[3-cyclohexyl-3-(trans-4-propoxy-cyclohexyl)-ureido]-thiazol-5-ylsulfanyl}-acetic acid was prepared using the method above, where the polymeric stabilizer was polyvinyl pyrrolidone and the surfactant stabilizer was sodium lauryl sulfate (SLS). The resulting nanosuspension had a 10% solid content, a mean particle size of 258.7 nm, a polydispersity index of 0.154 and a zeta potential of −58.3 mV.

### Example 7

A nanosuspension of {2-[3-cyclohexyl-3-(trans-4-propoxy-cyclohexyl)-ureido]-thiazol-5-ylsulfanyl}-acetic acid is prepared using the method above, where the polymeric stabilizer is polyvinyl sulfate and the surfactant stabilizer is sodium lauryl sulfate (SLS).

### Example 8

A nanosuspension of {2-[3-cyclohexyl-3-(trans-4-propoxy-cyclohexyl)-ureido]-thiazol-5-ylsulfanyl}-acetic acid is prepared using the method above, where the polymeric stabilizer is polyethylene glycol-polylactic acid (PEG-PLA) and the surfactant stabilizer is sodium lauryl sulfate (SLS).

### Example 9

A nanosuspension of {2-[3-cyclohexyl-3-(trans-4-propoxy-cyclohexyl)-ureido]-thiazol-5-ylsulfanyl}-acetic acid is prepared using the method above, where the polymeric stabilizer is gelatin and the surfactant stabilizer is sodium lauryl sulfate (SLS).

### Example 10

A nanosuspension of {2-[3-cyclohexyl-3-(trans-4-propoxy-cyclohexyl)-ureido]-thiazol-5-ylsulfanyl}-acetic acid is prepared using the method above, where the polymeric stabilizer is agar and the surfactant stabilizer is sodium lauryl sulfate (SLS).

### Example 11

A nanosuspension of {2-[3-cyclohexyl-3-(trans-4-propoxy-cyclohexyl)-ureido]-thiazol-5-ylsulfanyl}-acetic acid is prepared using the method above, where the polymeric stabilizer is hydroxypropylmethyl cellulose and the surfactant stabilizer is polysorbate 80.

### Example 12

A nanosuspension of {2-[3-cyclohexyl-3-(trans-4-propoxy-cyclohexyl)-ureido]-thiazol-5-ylsulfanyl}-acetic acid is prepared using the method above, where the polymeric stabilizer is hydroxypropylmethyl cellulose and the surfactant stabilizer is sodium docusate.

### Example 13

A nanosuspension of {2-[3-cyclohexyl-3-(trans-4-propoxy-cyclohexyl)-ureido]-thiazol-5-ylsulfanyl}-acetic acid is prepared using the method above, where the polymeric stabilizer is hydroxypropylmethyl cellulose and the surfactant stabilizer is sodium deoxycholate.

### Example 14

A nanosuspension of {2-[3-cyclohexyl-3-(trans-4-propoxy-cyclohexyl)-ureido]-thiazol-5-ylsulfanyl}-acetic acid is prepared using the method above, where the polymeric stabilizer is hydroxypropylmethyl cellulose and the surfactant stabilizer is vitamin E polyethylene glycol succinate.

### B. Preparation of Nanogranules

### General Experimental Procedure to Prepare Nanogranules of {2-[3-cyclohexyl-3-(trans-4-propoxy-cyclohexyl)-ureido]-thiazol-5-ylsulfanyl}-acetic acid (drug ingredient)

Solvent was removed from a nanosuspension obtained from Example 1 until the nanosuspension had a total weight of 1000 g with 10% w/w solid content. A redispersing agent (50 g, 5% w/w) was added to the nanosuspension and the mixture was stirred until redispersing agent was completely dissolved. A fluid bed was warmed to about 60° C. and a binder (800 g, 80% w/w) and filler (5 g, 5% w/w) were added to the fluid bed. The excipients were fluidized in the fluid bed to mix. The nanosuspension was top sprayed in the fluid bed while maintaining a product temperature of about 40° C. After top spraying is complete, the resulting granules were dried at 40° C. until less than 3% loss on drying (LOD) of the granules is obtained.

### Example 15

Nanogranules of {2-[3-cyclohexyl-3-(trans-4-propoxy-cyclohexyl)-ureido]-thiazol-5-ylsulfanyl}-acetic acid were prepared using the general method above, where the redispersing agent was mannitol, the binder was hydroxypropylmethyl cellulose and the filler was microcrystalline cellulose.

The nanoparticle size after granulation is shown in Table 4 below, with a comparison of particle size with and without redispersing agent.

TABLE 4
Particle size of reconstituted {2-[3-cyclohexyl-3-(trans-4-propoxy-
cyclohexyl)-ureido]-thiazol-5-ylsulfanyl}-acetic acid

| | Nanoparticle size (nm) | PDI |
|---|---|---|
| Before granulation | 252.1 | 0.172 |
| Reconstituted nanogranule | 479.3 | 0.385 |
| without redispersing agent | | |
| Reconstituted nanogranule | 399.6 | 0.322 |
| with redispersing agent | | |

### Example 16

Nanogranules of {2-[3-cyclohexyl-3-(trans-4-propoxy-cyclohexyl)-ureido]-thiazol-5-ylsulfanyl}-acetic acid are prepared using the general method above, where the redispersing agent is trehalose, the binder is hydroxypropylmethyl cellulose and the filler is microcrystalline cellulose.

### Example 17

Nanogranules of {2-[3-cyclohexyl-3-(trans-4-propoxy-cyclohexyl)-ureido]-thiazol-5-ylsulfanyl}-acetic acid are prepared using the general method above, where the redispersing agent is sorbitol, the binder is hydroxypropylmethyl cellulose and the filler is microcrystalline cellulose.

### Example 18

Nanogranules of {2-[3-cyclohexyl-3-(trans-4-propoxy-cyclohexyl)-ureido]-thiazol-5-ylsulfanyl}-acetic acid are prepared using the general method above, where the redispersing agent is lactose, the binder is hydroxypropylmethyl cellulose and the filler is microcrystalline cellulose.

### Example 19

Nanogranules of {2-[3-cyclohexyl-3-(trans-4-propoxy-cyclohexyl)-ureido]-thiazol-5-ylsulfanyl}-acetic acid are prepared using the general method above, where the redispersing agent is sucrose, the binder is hydroxypropylmethyl cellulose and the filler is microcrystalline cellulose.

### Example 20

Nanogranules of {2-[3-cyclohexyl-3-(trans-4-propoxy-cyclohexyl)-ureido]-thiazol-5-ylsulfanyl}-acetic acid are prepared using the general method above, where the redispersing agent is isomalt, the binder is hydroxypropylmethyl cellulose and the filler is microcrystalline cellulose.

### Example 21

Nanogranules of {2-[3-cyclohexyl-3-(trans-4-propoxy-cyclohexyl)-ureido]-thiazol-5-ylsulfanyl}-acetic acid are prepared using the general method above, where the redispersing agent is innulin, the binder is hydroxypropylmethyl cellulose and the filler is microcrystalline cellulose.

### Example 22

Nanogranules of {2-[3-cyclohexyl-3-(trans-4-propoxy-cyclohexyl)-ureido]-thiazol-5-ylsulfanyl}-acetic acid are prepared using the general method above, where the redispersing agent is dextran, the binder is hydroxypropylmethyl cellulose and the filler is microcrystalline cellulose.

### Example 23

Nanogranules of {2-[3-cyclohexyl-3-(trans-4-propoxy-cyclohexyl)-ureido]-thiazol-5-ylsulfanyl}-acetic acid are prepared using the general method above, where the redispersing agent is mannitol, the binder is hydroxypropylmethyl cellulose and the filler is dicalcium phosphate.

### Example 24

Nanogranules of {2-[3-cyclohexyl-3-(trans-4-propoxy-cyclohexyl)-ureido]-thiazol-5-ylsulfanyl}-acetic acid are prepared using the general method above, where the redispersing agent is mannitol, the binder is hydroxypropylmethyl cellulose and the filler is isomalt.

### Example 25

Nanogranules of {2-[3-cyclohexyl-3-(trans-4-propoxy-cyclohexyl)-ureido]-thiazol-5-ylsulfanyl}-acetic acid are prepared using the general method above, where the redispersing agent is mannitol, the binder is hydroxypropylmethyl cellulose and the filler is lactose.

### Example 26

Nanogranules of {2-[3-cyclohexyl-3-(trans-4-propoxy-cyclohexyl)-ureido]-thiazol-5-ylsulfanyl}-acetic acid are prepared using the general method above, where the redispersing agent is mannitol, the binder is hydroxypropylmethyl cellulose and the filler is mannitol.

### Example 27

Nanogranules of {2-[3-cyclohexyl-3-(trans-4-propoxy-cyclohexyl)-ureido]-thiazol-5-ylsulfanyl}-acetic acid are prepared using the general method above, where the redispersing agent is mannitol, the binder is hydroxypropylmethyl cellulose and the filler is starch.

### Example 28

Nanogranules of {2-[3-cyclohexyl-3-(trans-4-propoxy-cyclohexyl)-ureido]-thiazol-5-ylsulfanyl}-acetic acid are prepared using the general method above, where the redispersing agent is mannitol, the binder is hydroxypropylmethyl cellulose and the filler is trehalose.

### Example 29

Nanogranules of {2-[3-cyclohexyl-3-(trans-4-propoxy-cyclohexyl)-ureido]-thiazol-5-ylsulfanyl}-acetic acid are prepared using the general method above, where the redispersing agent is mannitol, the binder is hydroxypropylmethyl cellulose and the filler is sodium carbonate.

### Example 30

Nanogranules of {2-[3-cyclohexyl-3-(trans-4-propoxy-cyclohexyl)-ureido]-thiazol-5-ylsulfanyl}-acetic acid are prepared using the general method above, where the redispersing agent is mannitol, the binder is hydroxypropylmethyl cellulose and the filler is glucose.

### Example 31

Nanogranules of {2-[3-cyclohexyl-3-(trans-4-propoxy-cyclohexyl)-ureido]-thiazol-5-ylsulfanyl}-acetic acid are prepared using the general method above, where the redispersing agent is mannitol, the binder is hydroxypropylmethyl cellulose and the filler is hydroxypropylmethyl cellulose.

### Example 32

Nanogranules of {2-[3-cyclohexyl-3-(trans-4-propoxy-cyclohexyl)-ureido]-thiazol-5-ylsulfanyl}-acetic acid are prepared using the general method above, where the redispersing agent is mannitol, the binder is hydroxypropyl cellulose and the filler is microcrystalline cellulose.

### Example 33

Nanogranules of {2-[3-cyclohexyl-3-(trans-4-propoxy-cyclohexyl)-ureido]-thiazol-5-ylsulfanyl}-acetic acid are prepared using the general method above, where the redispersing agent is mannitol, the binder is polyvinyl pyrrolidone and the filler is microcrystalline cellulose.

### Example 34

Nanogranules of {2-[3-cyclohexyl-3-(trans-4-propoxy-cyclohexyl)-ureido]-thiazol-5-ylsulfanyl}-acetic acid are prepared using the general method above, where the redispersing agent is mannitol, the binder is polyvinyl alcohol and the filler is microcrystalline cellulose.

### Example 35

Nanogranules of {2-[3-cyclohexyl-3-(trans-4-propoxy-cyclohexyl)-ureido]-thiazol-5-ylsulfanyl}-acetic acid are prepared using the general method above, where the redispersing agent is mannitol, the binder is polyvinyl caprolactam and the filler is microcrystalline cellulose.

### Example 36

Nanogranules of {2-[3-cyclohexyl-3-(trans-4-propoxy-cyclohexyl)-ureido]-thiazol-5-ylsulfanyl}-acetic acid are prepared using the general method above, where the redispersing agent is mannitol, the binder is gelatin and the filler is microcrystalline cellulose.

### Example 37

Nanogranules of {2-[3-cyclohexyl-3-(trans-4-propoxy-cyclohexyl)-ureido]-thiazol-5-ylsulfanyl}-acetic acid are prepared using the general method above, where the redispersing agent is mannitol, the binder is pregelatinized starch and the filler is microcrystalline cellulose.

### C. Preparation of Spray-Dried Nanoparticles

General Experimental Procedure to Prepare Spray-Dried Nanoparticles of {2-[3-cyclohexyl-3-(trans-4-propoxy-cyclohexyl)-ureido]-thiazol-5-ylsulfanyl}-acetic acid (drug ingredient):

Solvent was removed from a nanosuspension obtained from Example 1 until the nanosuspension had a total weight of 1000 g with 10% w/w solid content. A redispersing agent (50 g, 5% w/w) was added to the nanosuspension and the mixture was stirred until the redispersing agent was completely dissolved. The nanosuspension was spray dried using a spray dryer with an inlet temperature of 90±10° C. and outlet temperature of 50±10° C. After all of the nanosuspension was spray dried, the dried nanoparticles were collected from cyclone of the spray drier (Buchi Mini Spray Dryer B-290, Buchi Labortechnik AG, Flawil, Switzerland). TGA analysis of the resulting spray dried nanoparticles gave a moisture content of about 0.1%. The particle size of the spray-dried nanoparticles was characterized using a Melvern Zetasizer ZS (Model Zen3600, by Malvern Instruments, Ltd., Worcestershire, United Kingdom).

### Example 38

Spray-dried nanoparticles were prepared using the general procedure above, where the redispersing agent was mannitol. Table 5 shows the particle size of nanoparticles of {2-[3-cyclohexyl-3-(trans-4-propoxy-cyclohexyl)-ureido]-thiazol-5-ylsulfanyl}-acetic acid before and after spray drying.

TABLE 5
Particle size of spray-dried nanoparticles of {2-[3-cyclohexyl-3-(trans-
4-propoxy-cyclohexyl)-ureido]-thiazol-5-ylsulfanyl}-acetic acid

| | Nanoparticle size (nm) | PDI |
|---|---|---|
| Before spray drying | 222 | 0.112 |
| (nanosuspension) | | |
| spray dried nanoparticle | 258 | 0.219 |

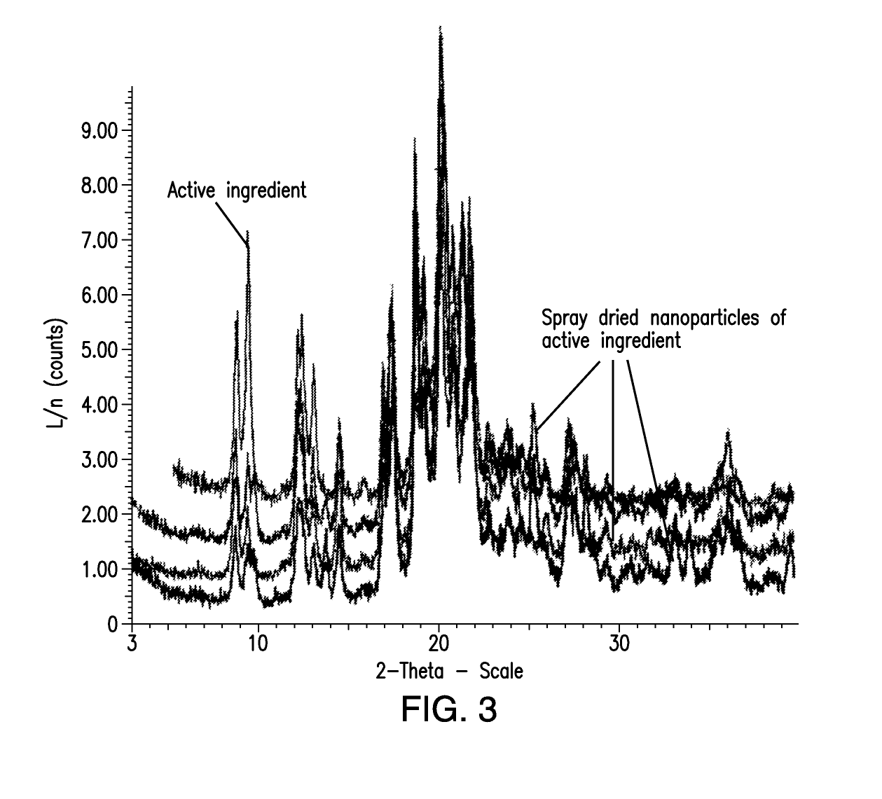

FIG. 3 shows X-ray powder diffraction (XRPD) patterns of {2-[3-cyclohexyl-3-(trans-4-propoxy-cyclohexyl)-ureido]-thiazol-5-ylsulfanyl}-acetic acid and spray-dried nanoparticles of {2-[3-cyclohexyl-3-(trans-4-propoxy-cyclohexyl)-ureido]-thiazol-5-ylsulfanyl}-acetic acid. The crystal structure of {2-[3-cyclohexyl-3-(trans-4-propoxy-cyclohexyl)-ureido]-thiazol-5-ylsulfanyl}-acetic acid was not changed during spray drying.

Stability of the active ingredient after being subjected to spray drying is shown in Table 6 below. Spray drying did not result in significant degradation of the active ingredient.

TABLE 6
Degradation profile of spray dried nanoparticles of {2-[3-cyclohexyl-3-
(trans-4-propoxy-cyclohexyl)-ureido]-thiazol-5-ylsulfanyl}-acetic acid

| | | | | | | | | | | | | Drug |
|---|---|---|---|---|---|---|---|---|---|---|---|---|
| | Degradant Profile | | | | | | | | | | | Ingredient |
| Sample | a | b | c | d | e | f | g | h | i | j | k | % Assay |
| % | 0.17 | 0 | 0 | 0.02 | 0.09 | 0.01 | 0.04 | 0.01 | 0.03 | 0.04 | 0.02 | 99.53 |

### D. Solid dosage forms of {2-[3-cyclohexyl-3-(trans-4-propoxy-cyclohexyl)-ureido]-thiazol-5-ylsulfanyl}-acetic acid

### Example 39

### Preparation of Capsules Containing Spray Dried Nanoparticles

Spray dried nanoparticles prepared in Example 38 (424 mg, containing {2-[3-cyclohexyl-3-(trans-4-propoxy-cyclohexyl)-ureido]-thiazol-5-ylsulfanyl}-acetic acid 0.47 g/g) was blended with meglumine (100 mg) in a V-blender for 15 minutes, and then tested for blend uniformity. The blend was then charged into a powder station of a capsule filling machine. After adjusting the capsule fill weight based on the composition, the blend was filled into gelatin capsules.

### Example 40

### Preparation of Capsules Containing Nanogranules

A mixture of isomalt (106 mg, Galen IQ 800), polyvinyl pyrrolidone (20 mg, PVP K30) and meglumine (150 mg) was screened through a 20 mesh hand screen. The screened mixture was added to a fluid bed granulator heated to 50° C., and the nanosuspension of {2-[3-cyclohexyl-3-(trans-4-propoxy-cyclohexyl)-ureido]-thiazol-5-ylsulfanyl}-acetic acid obtained in Example 1 (about 2224 g) was top-sprayed onto the substrate. The product temperature was maintained at 40° C. during granulation. After granulation was completed, the nanogranules were dried until the moisture content was less than 3%. The nanogranules were discharged and then stored in a refrigerator for at least 12 hours, and then brought to room temperature. The nanogranules (500 mg) were then charged into a powder station of a capsule filling machine. After adjusting the capsule fill weight based on the composition, the nanogranules were filled into gelatin capsules (size AA EL, Grey Opaque).

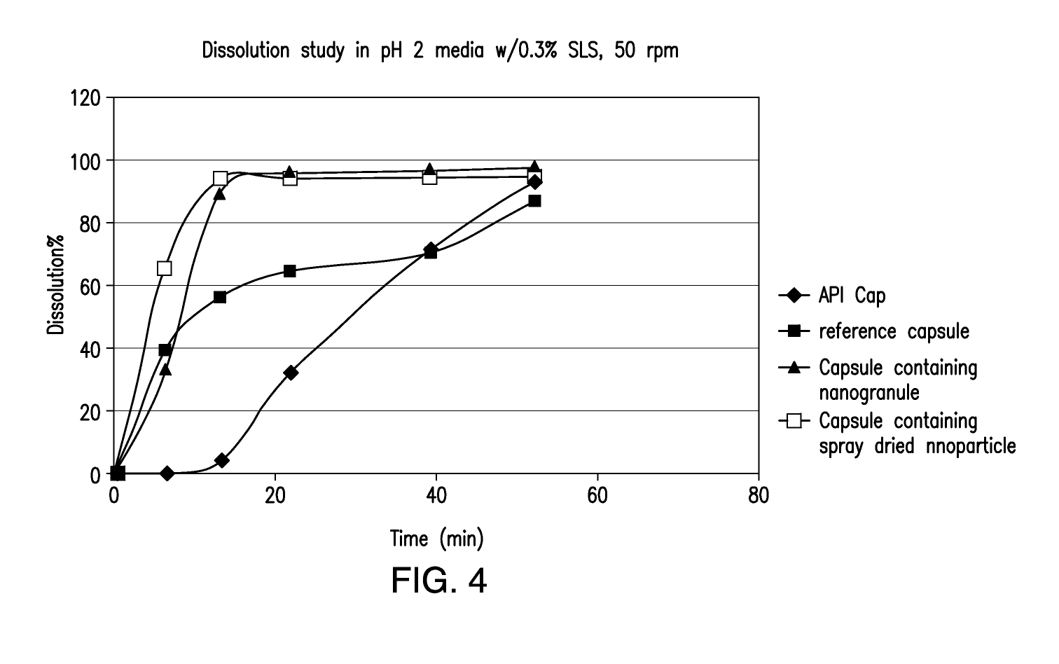

FIG. 4 shows the dissolution of: a capsule containing only micronized {2-[3-cyclohexyl-3-(trans-4-propoxy-cyclohexyl)-ureido]-thiazol-5-ylsulfanyl}-acetic acid (API Cap); a capsule containing granules formulated with micronized {2-[3-cyclohexyl-3-(trans-4-propoxy-cyclohexyl)-ureido]-thiazol-5-ylsulfanyl}-acetic acid (reference capsule) a capsule containing spray-dried nanoparticles of {2-[3-cyclohexyl-3-(trans-4-propoxy-cyclohexyl)-ureido]-thiazol-5-ylsulfanyl}-acetic acid obtained from Example 39, and a capsule containing nanogranules of {2-[3-cyclohexyl-3-(trans-4-propoxy-cyclohexyl)-ureido]-thiazol-5-ylsulfanyl}-acetic acid obtained from Example 40.

The dissolutions were conducted in an aqueous medium of 0.3% w/v sodium laurel sulfate in 0.01 N HCl, pH 2, pedal method, 50 rpm. The dissolution was performed at 37° C. for up to 60 minutes, and dissolution sampling was performed at 15, 30, 45 and 60 minutes. Drug release was analyzed using HPLC.

### Example 41

### Preparation of Tablets Containing Nanogranules

A mixture of isomalt (106 mg, Galen IQ 800), polyvinyl pyrrolidone (20 mg, PVP K30) and meglumine (150 mg) was screened through a 20 mesh hand screen. The screened mixture was added to a fluid bed granulator heated to 50° C., and the nanosuspension of {2-[3-cyclohexyl-3-(trans-4-propoxy-cyclohexyl)-ureido]-thiazol-5-ylsulfanyl}-acetic acid obtained in Example 1 (about 2224 g) was top-sprayed onto the substrate. The product temperature was maintained at 40° C. during granulation. After granulation was completed, the nanogranules were dried until the moisture content was less than 3%. The nanogranules were discharged and then stored in a refrigerator.

The nanogranules were brought to room temperature and then milled to obtain uniform particle size. The milled granules (500 mg) were screened through a 20 mesh hand screen then added to a V-blender. A mixture of croscarmellose sodium (18 mg), isomalt (74 mg) was screened through a 20 mesh hand screen and added to the V-blender. The mixture was blended for about 15 minutes. Talc (5 mg) was screened through a 20 mesh hand screen and then added to the V-blender. The mixture was blended for about 5 minutes. Magnesium stearate (3 mg) was screened through a 20 mesh hand screen and added to the V-blender. The mixture was blended for 5 minutes. The blended mixture was then discharged. The blended mixture was added into the hopper of a tablet press, and was compressed to form a tablet with a weight of 600 mg and hardness of 8 to 12 kiloponds (kp).

### Example 42

### A Single Dose Study Conducted in Male Beagle Dogs

Various 100 mg {2-[3-cyclohexyl-3-(trans-4-propoxy-cyclohexyl)-ureido]-thiazol-5-ylsulfanyl}-acetic acid oral formulations were administered in single doses to male beagle dogs on Day 1 and Day 11. Measurable plasma samples were obtained and analyzed at 0.5 hours to 24 hours for concentrations of {2-[3-cyclohexyl-3-(trans-4-propoxy-cyclohexyl)-ureido]-thiazol-5-ylsulfanyl}-acetic acid by a validated LC-MS/MS assay (Internal Standards: deuterated compounds; Sample Preparation: liquid extraction; Sample Volume: 1 mL; Calibration Range: 1.00-1.000 ng/mL; Ionization: Turbo IonSpray).

The subjects in the study were administered the following formulations:

A. 10 mL aqueous solution of 10 mg/mL {2-[3-cyclohexyl-3-(trans-4-propoxy-cyclohexyl)-ureido]-thiazol-5-ylsulfanyl}-acetic acid and 1% meglumine

B. Capsules containing nanogranules having 100 mg {2-[3-cyclohexyl-3-(trans-4-propoxy-cyclohexyl)-ureido]-thiazol-5-ylsulfanyl}-acetic acid,

C. Spray-dried nanoparticle capsules containing 100 mg {2-[3-cyclohexyl-3-(trans-4-propoxy-cyclohexyl)-ureido]-thiazol-5-ylsulfanyl}-acetic acid, and

D. Capsules containing granules formulated with 100 mg micronized {2-[3-cyclohexyl-3-(trans-4-propoxy-cyclohexyl)-ureido]-thiazol-5-ylsulfanyl}-acetic acid (reference capsule).

The mean pharmacokinetic parameters observed after administration of a single dose of the above formulations are shown below in Table 7.

TABLE 7
Pharmacokinetic Parameters on Day 1 and Day 11
Following Oral Administration of 100 mg active
ingredient in Various Formulations to Male Dogs

| Formulation | Cmax (ng/mL) | Tmax (hr) | AUC_{0-∞} (ng*hr/mL) |
|---|---|---|---|
| A | 8870 | 0.667 | 13686 |
| B | 6730 | 1.17 | 14196 |
| C | 6657 | 0.833 | 11532 |
| D | 2680 | 2.33 | 7488 |

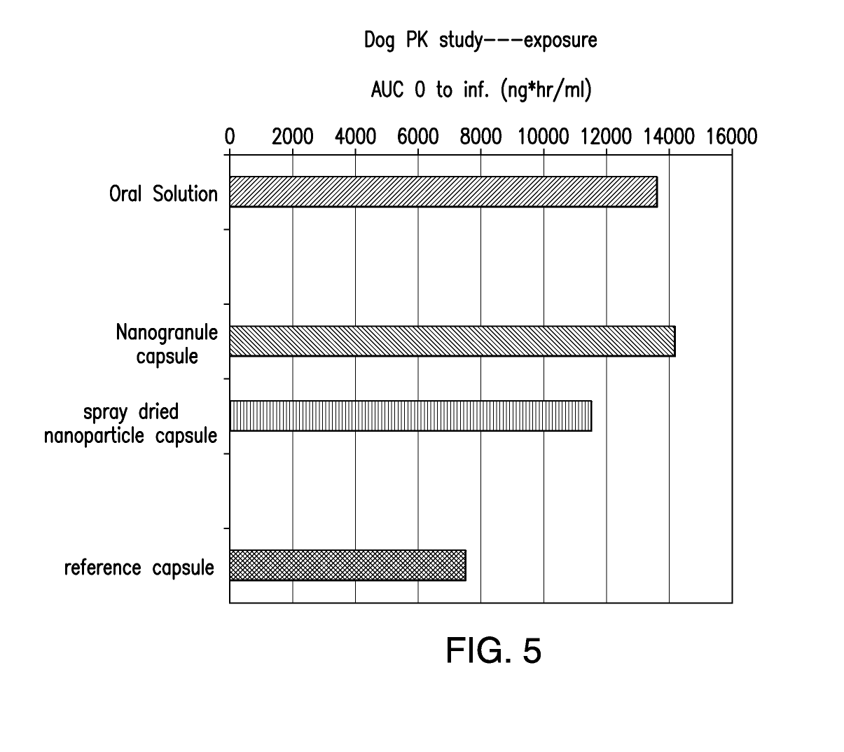

FIG. 5 shows the in vivo exposure (AUC_{0-∞}) of these various formulations in beagle dogs.

### Example 43

### Single-Dose Study Conducted in Healthy Subjects

Healthy subjects were randomized into different groups (A, B and C) in a single-center, randomized, open-label, 4-way cross-over study with 7-day washout period. Measurable plasma samples were obtained and analyzed at 1 hour to 24 hours for concentrations of {2-[3-cyclohexyl-3-(trans-4-propoxy-cyclohexyl)-ureido]-thiazol-5-ylsulfanyl}-acetic acid by a validated assay.

The subjects were administered the following formulations under fasted conditions with water:

A. 4×200 mg {2-[3-cyclohexyl-3-(trans-4-propoxy-cyclohexyl)-ureido]-thiazol-5-ylsulfanyl}-acetic acid capsule (reference)

B. 4×200 mg capsules of spray-dried nanoparticles of {2-[3-cyclohexyl-3-(trans-4-propoxy-cyclohexyl)-ureido]-thiazol-5-ylsulfanyl}-acetic acid

C. 4×200 mg capsules of {2-[3-cyclohexyl-3-(trans-4-propoxy-cyclohexyl)-ureido]-thiazol-5-ylsulfanyl}-acetic acid nanogranules.

### Formulation A

### Reference

Microgranules of Formulation A was prepared with the following ingredients:

| Ingredient | % w/w | Theor. wt (mg/g) |
|---|---|---|
| Active Ingredient | 65.60 | 657.0 |
| Microcrystalline Cellulose, NF | 11.50 | 115.0 |
| (Ph. Eur. (Avicel PH101) | | |
| Lactose Monohydrate | 12.29 | 122.9 |
| Croscarmellose sodium, NF (Ac-Di-Sol) | 7.56 | 75.6 |
| Polysorbate 80, NF | 2.37 | 23.7 |
| Hydropropyl Methylcellulose E3 LV | 0.59 | 5.9 |
| premium | | |
| Sterile Water for irrigation | N/A | N/A |
| Granule Total, 200 mg active | 100 | 1000 |
| ingred./304.4 mg | | |

A master blend having the following ingredients was then prepared from the microgranules:

| Ingredient | % w/w | Theor. wt (mg/g) |
|---|---|---|
| Granule Total, 200 mg active | 84.56 | 845.6 |
| ingred./304.4 mg | | |
| Microcrystalline Cellulose, NF (Ph. Eur. | 4.94 | 49.4 |
| (Avicel PH101) | | |
| Croscarmellose sodium, NF (Ac-Di-Sol) | 5.00 | 50.0 |
| Pregelatinized Starch, NF (Starch 1500) | 5.00 | 50.0 |
| Magnesium Stearate, NF (Hyqual 2257) | 0.50 | 5.0 |
| Active Ingred. Master Blend, | 100 | 1000 |
| 200 mg/360 mg | | |

Capsules (gelatin capsules, size 0, gray opaque, 96 mg) were then filled with 180 mg of the active ingredient master blend, 200 mg/360 mg.

### Formulation B

### Capsules with Spray Dried Nanoparticles

A nanoparticle suspension composition with 10% solids was prepared as described in Example 2, except using the following modified amounts for each ingredient:

| Ingredient | % w/w | Theor. wt (mg/g) |
|---|---|---|
| Active Ingredient | 10.0 | 9.0 |
| Hydropropyl Cellulose | 0.2 | 0.2 |
| Sodium Lauryl Sulfate | 1 | 0.9 |
| Purified Water, USP | N/A | 89.9 |
| Nanoparticle Suspension, 10% solids | N/A | 100 |

Using the procedure of Example 38, the nanoparticle suspension was spray-dried to form spray-dried nanoparticles, where the redispersing agent was mannitol in a modified amount of 90 g (9% w/w). The resulting spray dried nanoparticles contained 0.47 g of active ingredient per gram of total dry weight. A master blend was then formed by combining the spray dried nanoparticles (80.9%) with meglumine (19.1%), to form a master blend having 200 mg of active ingredient per 524 mg total dry weight. Capsules (gelatin capsules, size AA EL, gray opaque, 168 mg) were then filled with 524 mg of the active ingredient master blend, 200 mg/524 mg.

### Formulation C

### Capsules with Nanogranules

Nanoparticle granules were prepared using the general procedure in Example 40, except the nanoparticle suspension was obtained in Formulation B above, and each ingredient amount was modified as follows:

| Ingredient | % w/w | Theor. wt (mg/g) |
|---|---|---|
| Nanoparticle Suspension, 10% solids | 44.8 | 448 |
| Isomalt (GalenIQ 800) | 21.2 | 212 |
| Meglumine | 30 | 300 |
| Polyvinyl Pyrrolidone (PVP K30) | 4 | 40 |
| Nanoparticle Granules, 200 mg/500 mg | 100 | 1000 |

Capsules (gelatin capsules, size AA EL, gray opaque, 168 mg) were then filled with 500 mg of the nanoparticle granules, 200 mg/500 mg.

The mean pharmacokinetic parameters observed after administration of a single dose of the above formulations are shown below in Table 8.

TABLE 8
Summary Pharmacokinetic Parameters Following
Oral Administration in Healthy Subjects

| Formulation | Cmax (ng/mL) | Tmax (hr) | AUC_{0-∞} (ng*hr/mL) |
|---|---|---|---|
| A | 1382 | 1.5 | 4705 |
| B | 5802 | 1.5 | 10347 |
| C | 5606 | 1.0 | 10231 |

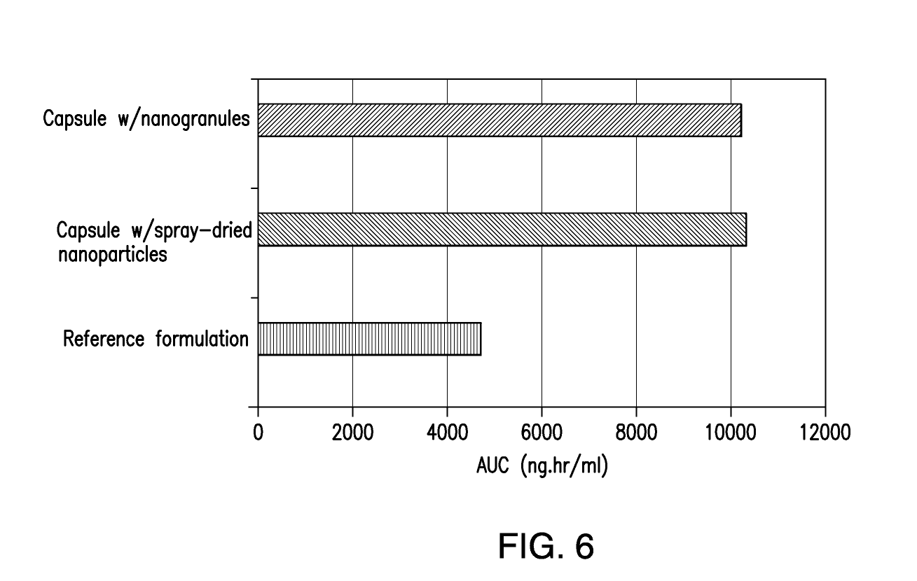

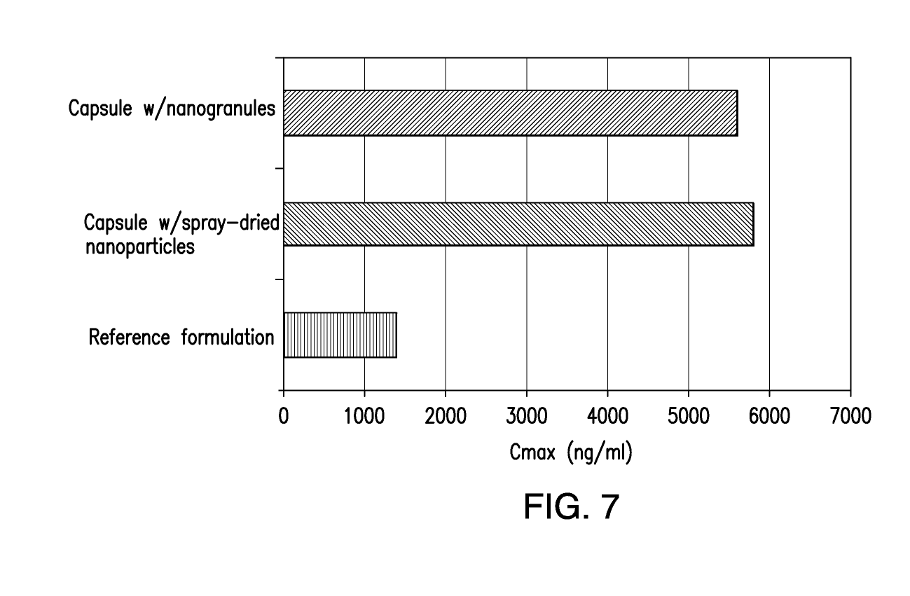

FIGS. 6 and 7 show the in vivo exposure (AUC and Cₘₐₓ respectively) in the three groups.

### Example 44

### Multiple Dose Study in Patients with Type 2 Diabetes Mellitus

Patients with Type 2 diabetes mellitus were randomized into different groups (A, B and C) in a multi-center, randomized, double-blind, parallel-group, multiple-dose study. The patients were administered the following formulations:

A. Single oral dose of {2-[3-cyclohexyl-3-(trans-4-propoxy-cyclohexyl)-ureido]-thiazol-5-ylsulfanyl}-acetic acid (lx 200 mg capsule) on Day 1 followed by multiple oral doses of {2-[3-cyclohexyl-3-(trans-4-propoxy-cyclohexyl)-ureido]-thiazol-5-ylsulfanyl}-acetic acid (1×200 mg capsule) twice daily (BID) (reference).

B. Single oral dose of 800 mg {2-[3-cyclohexyl-3-(trans-4-propoxy-cyclohexyl)-ureido]-thiazol-5-ylsulfanyl}-acetic acid (4×200 mg capsule) on Day 1 followed by multiple oral doses of {2-[3-cyclohexyl-3-(trans-4-propoxy-cyclohexyl)-ureido]-thiazol-5-ylsulfanyl}-acetic acid 800 mg (4×200 mg capsule) once daily (QD).

C. Single oral dose of 800 mg {2-[3-cyclohexyl-3-(trans-4-propoxy-cyclohexyl)-ureido]-thiazol-5-ylsulfanyl}-acetic acid (4×200 mg capsule) on Day 1 followed by multiple oral doses of {2-[3-cyclohexyl-3-(trans-4-propoxy-cyclohexyl)-ureido]-thiazol-5-ylsulfanyl}-acetic acid 800 mg (4×200 mg capsule) twice daily (BID).

The mean pharmacokinetic parameters observed after administration of a single dose of the above formulations are shown below in Table 9.

TABLE 9
Summary Pharmacokinetic Parameters After 42 days Following Oral
Administration in Patients with Type 2 Diabetes Mellitus

| Formulation | Cmax (ng/mL) | Tmax (hr) | AUC₀₋₂₄ (ng*hr/mL) |
|---|---|---|---|
| A | 1047 | 1 | 3825 |
| B | 10802 | 1 | 16218 |
| C | 9794 | 1 | 29407 |

What is claimed is:

1. A pharmaceutical composition comprising nanoparticles and one or more alkalizers, wherein the nanoparticles have a mean diameter between about 0.5 nm to about 1000 nm, have a polydispersity index of about 0.001 to about 0.400 and comprise {2-[3-cyclohexyl-3-(trans-4-propoxy-cyclohexyl)-ureido]-thiazol-5-ylsulfanyl}-acetic acid or a pharmaceutically acceptable salt thereof.

2. The pharmaceutical composition of claim 1, wherein the nanoparticles further comprise one or more redispersing agents.

3. The pharmaceutical composition of claim 1, wherein the nanoparticles have a mean diameter between about 200 nm to about 400 nm.

4. The pharmaceutical composition of claim 3, wherein the nanoparticles have a polydispersity index of about 0.001 to about 0.300.

5. The pharmaceutical composition of claim 1, wherein the one or more alkalizers are selected from the group consisting of meglumine, sodium carbonate, potassium carbonate, calcium carbonate, magnesium oxide, calcium hydroxide, sodium hydroxide, potassium hydroxide, diethanolamine, potassium bicarbonate, potassium citrate, sodium borate, sodium citrate, triethanolamine and a mixture thereof.

6. The pharmaceutical composition of claim 5, wherein the one or more alkalizer is meglumine.

7. The pharmaceutical composition of claim 2, wherein the one or more redispersing agents are selected from the group consisting of mannitol, trehalose, xylitol, lactose, sucrose, sorbitol, dextran, lactitol, maltitol, erythritol, threitol, arabitol, ribitol, galactitol, fucitol, iditol, inocitol, velomitol, isomalt, inulin and a mixture thereof.

8. The pharmaceutical composition of claim 1, wherein the nanoparticles are formed by removing solvent from a nanoparticle suspension comprising:
(a) {2-[3-cyclohexyl-3-(trans-4-propoxy-cyclohexyl)-ureido]-thiazol-5-ylsulfanyl}-acetic acid or a pharmaceutically acceptable salt thereof,
(b) one or more stabilizers,
(c) one or more redispersing agents, and
(d) one or more solvents.

9. The pharmaceutical composition of claim 8, wherein the one or more solvents are selected from the group consisting of water, methanol, heptane, propanol, isopropanol, acetic acid, acetone, ethyl acetate, ethanol and a mixture thereof.

10. The pharmaceutical composition of claim 8, wherein the one or more stabilizers are selected from the group consisting of a polymeric stabilizer, a surfactant stabilizer and a mixture thereof.

11. The pharmaceutical composition of claim 8, wherein the nanoparticle suspension has a zeta potential of greater than about 30 mV or less than about −30 mV.

12. The pharmaceutical composition of claim 1, further comprising one or more of a binder, a filler, a diluent, a disintegrant or a mixture thereof.

13. The pharmaceutical composition of claim 1, wherein the composition comprises about 800 mg {2-[3-cyclohexyl-3-(trans-4-propoxy-cyclohexyl)-ureido]-thiazol-5-ylsulfanyl}-acetic acid, or a pharmaceutically acceptable salt thereof, and provides an in vivo plasma profile comprising:
(i) a mean Cₘₐₓ of less than about 6000 ng/mL
(ii) a mean AUC_{0-∞} of more than about 5000 ng·hr/mL, and
(iii) a mean Tₘₐₓ of about 1 or more hours.

14. The pharmaceutical composition of claim 13, wherein the mean AUC_{0-∞} is more than about 7500 ng·hr/mL.

15. The pharmaceutical composition of claim 1, wherein the pharmaceutical composition releases {2-[3-cyclohexyl-3-(trans-4-propoxy-cyclohexyl)-ureido]-thiazol-5-ylsulfanyl}-acetic acid, or a pharmaceutically acceptable salt thereof at a rate of more than about 80% of the drug within the first 15 minutes following administration of the formulation to a patient in need thereof.

16. A pharmaceutical composition comprising nanoparticles, one or more alkalizers and one or more redispersing agents, wherein the nanoparticles have a mean diameter between about 0.5 nm to about 1000 nm and comprise {2-[3-cyclohexyl-3-(trans-4-propoxy-cyclohexyl)-ureido]-thiazol-5-ylsulfanyl}-acetic acid or a pharmaceutically acceptable salt thereof.

17. A method of treating type II diabetes comprising administering to a patient in need thereof a pharmaceutical composition comprising nanoparticles and one or more alkalizers, wherein the nanoparticles have a mean diameter between about 0.5 nm to about 1000 nm, have a polydispersity index of about 0.001 to about 0.400 and comprise a therapeutically effective amount of {2-[3-cyclohexyl-3-(trans-4-propoxy-cyclohexyl)-ureido]-thiazol-5-ylsulfanyl}-acetic acid or a pharmaceutically acceptable salt thereof.

18. The method of claim 17, wherein the composition comprises about 800 mg {2-[3-cyclohexyl-3-(trans-4-propoxy-cyclohexyl)-ureido]-thiazol-5-ylsulfanyl}-acetic acid, or a pharmaceutically acceptable salt thereof, and provides an in vivo plasma profile comprising:
(i) a mean Cₘₐₓ of less than about 6000 ng/mL
(ii) a mean AUC_{0-∞} of more than about 5000 ng·hr/mL, and
(iii) a mean Tₘₐₓ of about 1 or more hours.

19. The method of claim 18, wherein the mean AUC_{0-∞} is more than about 7500 ng·hr/mL.

20. A method of improving glycemic control comprising administering to a patient in need thereof a pharmaceutical composition comprising nanoparticles and one or more alkalizers, wherein the nanoparticles have a mean diameter between about 0.5 nm to about 1000 nm, have a polydispersity index of about 0.001 to about 0.400 and comprise a therapeutically effective amount of {2-[3-cyclohexyl-3-(trans-4-propoxy-cyclohexyl)-ureido]-thiazol-5-ylsulfanyl}-acetic acid or a pharmaceutically acceptable salt thereof.

21. The method of claim 20, wherein the composition comprises about 800 mg {2-[3-cyclohexyl-3-(trans-4-propoxy-cyclohexyl)-ureido]-thiazol-5-ylsulfanyl}-acetic acid, or a pharmaceutically acceptable salt thereof, and provides an in vivo plasma profile comprising:
(i) a mean Cₘₐₓ of less than about 6000 ng/mL
(ii) a mean AUC_{0-∞} of more than about 5000 ng·hr/mL, and
(iii) a mean Tₘₐₓ of about 1 or more hours.

22. The method of claim 21, wherein the mean AUC_{0-∞} is more than about 7500 ng·hr/mL.
